(19) United States
(12) Patent Application Publication
(10) Pub. No.: US 2004/0106169 A1
(43) Pub. Date: Jun. 3, 2004

(54) System and method for metabolyte neuronal network analysis

(76) Inventor: Daron G. Evans, Dallas, TX (US)

Correspondence Address: CHALKER FLORES, LLP, 12700 PARK CENTRAL, STE. 455, DALLAS, TX, 75251, US

(21) Appl. No.: 10/371,008

(22) Filed: Feb. 20, 2003

Related U.S. Application Data
(60) Provisional application No. 60/430,409, filed on Dec. 2, 2002.

(51) Int. Cl. G01N 33/48; C12M 1/00

(52) U.S. Cl. 435/40.5; 435/283.1

(57) ABSTRACT

The present invention provides a system and method for testing the neuronal effects of a compound and its metabolites. The system (100) includes a microelectrode array (102), a data capture unit (108) communicably coupled to the microelectrode array (104), a processor (110) communicably coupled to the data capture unit (108) and one or more input/output devices (112) communicably coupled to the processor (110). The microelectrode array (102) is capable of supporting genetically modified neuronal cells (104) and measuring neuronal activity. The testing medium containing the compound and the metabolites is extracted from hepatocyte cells (106). The method (400) determines the effects of the metabolites of a sample compound on neuronal cells by exposing a sample compound to hepatocyte cells (406), extracting medium from the exposed cells (408) and exposing the extracted medium to neuronal cells on a microelectrode array (410). The effects of a sample compound and its metabolites versus the effects of a sample compound alone can be determined from a comparison of the data (406).

### TECHNICAL FIELD OF THE INVENTION

The present invention relates in general to the field of action potential analysis, and more particularly, to the use of advanced neuronal networks detection techniques for the detailed analysis of neuronal signal transduction pathways and their use for large-scale reproducible analysis.

### BACKGROUND OF THE INVENTION

This application claims priority to U.S. Provisional Patent Application Serial No. 60/430,409, filed Dec. 2, 2002. Without limiting the scope of the invention, the background of the invention is described in connection with the recording and analysis of neuronal action potentials using substrate integrated, thin film electrodes, as an example.

The first recordings of neuronal action potentials using substrate integrated, thin film electrodes were made as early as 1977 (Gross, et al. 1977). Subsequent research has led to multi-channel investigations of network dynamics and their applications. Indium-tin oxide was introduced later as a viable microelectrode material and was designed and tested for recording in life support chambers (Gross and Schwalm, 1995). These networks were used to explore stimulation of networks through the recording electrodes (Gross et al., 1994).

Linked dual, age-matched neuronal networks have been grown on microelectrode arrays with for possible uses as biosensors (Gross et al., 1995). A practical and realistic use of neural networks is in their application as physiological function deficit detectors. Due to electrophysiological mechanisms, neurons represent efficient transducers for detecting and recording the dynamics of cell death, receptor-ligand interactions, alterations in metabolism, cell signal transduction cascade events, and generic membrane perforation processes. As such, mammalian networks in culture, devoid of extra-neuronal homeostatic protection mechanisms, function as reliable and highly sensitive detectors of any toxicant capable of interfering with autonomic life support, neuromuscular functions, and even behavior.

Although single neurons are often vulnerable and unreliable, networks of neurons may be used to form robust, fault-tolerant, spontaneously active dynamic systems with high sensitivity to their chemical environment. Networks in culture generate response profiles that are concentration and substance specific and react to a broad range of compounds. Pharmacologically and toxicologically, neuronal networks are representative of the parent tissue.

### SUMMARY OF THE INVENTION

The present invention provides a system and method for testing the neuronal effects of a compound and/or its hepatic metabolites using a system that includes generally a microelectrode array, a data capture unit communicably coupled to the microelectrode array, a processor communicably coupled to the data capture unit and one or more input/output devices communicably coupled to the processor. The microelectrode array, which may be a MEA detector, is capable of supporting wild-type or genetically modified neuronal cells and measuring neuronal activity. The microelectrode array may also be a chamber having a fluid input connected to a perfusion system. The processor, which can be a computer, compares the neuronal activity of the neuronal cells in the presence and absence of the compound and in the presence of medium extracted from a hepatocyte culture in the presence and absence of the compound.

The system may also include a first and second chamber. The first chamber may be the microelectrode array. The neuronal cells may be from a 12-16 day old embryo of an animal, which could be a wild-type mouse or a genetically-modified mouse. The neuronal cells can be selected from the frontal cortex, the auditory cortex, the visual cortex, the hippocampus or the spinal cord. Furthermore, the neuronal cells may include one or more types of neuronal cells. In addition, the neuronal cells may be isolated from and form a neural tissue. The hepatocyte cells can be from a mature animal, a cell clone, a cell line (e.g., an immortalized human cell line) or combinations thereof. The hepatocytes may be isolated from wild-type or genetically-modified animals and may be obtained from any stage of gestation or age.

In addition, the present invention provides a method of determining the effects of a compound sample and/or the hepatic metabolites of the compound on neuronal cells in accordance with the present invention. In one example, separate cultures of hepatocyte cells are grown in separate chambers with similar cell counts, possibly in mono-layers. The compound sample is exposed to a hepatocyte culture. A sample of cell culture medium is extracted from hepatocyte cultures, which is exposed to the compound sample. Portions of the extracted hepatocyte medium are exposed to the neuronal cells. The effects of the extracted medium on the neuronal cells are measured to determine the effects of a compound sample and the metabolites of the compound sample on the neuronal cells.

The present invention also provides a method of determining the effects of hepatocyte cell culture medium on neuronal cells in accordance with the present invention is shown. The culture medium, often referred to also as a hepatic or hepatocyte supernatant, may or may not be cell-free. For example, to obtain separate culture medium from hepatocytes, the cells are grown in separate chambers with similar cell counts, possibly in mono-layers. A sample of cell culture medium is extracted from hepatocyte cultures that are not exposed to the compound sample. Portions of the extracted hepatocyte medium are exposed to the neuronal cells. The effects of the extracted medium on the neuronal cells are measured to determine the effects of a hepatocyte medium on the neuronal cells. The effect measured is used as a baseline for the measured effect on neuronal cells from hepatocyte medium that was exposed to a compound and/or its metabolites.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 is a block diagram of a system in accordance with the present invention;

FIGS. 2A, 2B, 3A and 3B illustrate typical microelectrode arrays that can be used in connection with the present invention;

FIG. 4 is a flow chart illustrating a process to determine the effects of a compound and a compound's metabolites on neuronal cells in accordance with the present invention;

FIG. 5 is a flow chart illustrating a testing method to determine the effects of a compound and its metabolites on neuronal cells in accordance with the present invention;

FIG. 6 is a flow chart outlining the basic steps for the testing method;

FIG. 7 is a flow chart describing the procedure to prepare the neural cell culture medium;

FIG. 8 is a flow chart describing the procedure to prepare the dissecting buffer;

FIG. 9 is a flow chart describing the procedure to prepare the other solutions (cell adhesion and enzyme solutions);

FIG. 10 is a flow chart describing the procedure to create the microelectrode array (MEA) substrate;

FIG. 11 is a flow chart describing the procedure to create the electrodes on the MEA substrate;

FIG. 12 is a flow chart describing the procedure to prepare the MEA for nerve cell culturing;

FIG. 13 is a flow chart describing the cell culturing procedure to prepare neural cell cultures;

FIG. 14 is a flow chart describing the procedure to nurture and care for the neural cell cultures;

FIG. 15 is a flow chart describing the procedure to prepare the hepatocyte cell culture medium;

FIG. 16 is a flow chart describing the procedure to prepare the culture flask for hepatocyte cell cultures;

FIG. 17 is a flow chart describing the cell culturing procedure to prepare hepatocyte cell cultures;

FIG. 18 is a flow chart describing the procedure to nurture and care for the hepatocyte cell cultures;

FIG. 19 is a flow chart describing the procedure to generate the metabolites of a compound;

FIG. 20 is a flow chart outlining the basic steps in the metabolite testing procedure;

FIG. 21 is a flow chart describing the procedure to select the cell culture to be used for testing;

FIG. 22 is a flow chart describing the procedure to autoclave the testing chamber;

FIG. 23 is a flow chart describing the procedure to assemble the testing chamber;

FIG. 24 is a flow chart describing the procedure to set up the testing station;

FIG. 25 is a flow chart describing the procedure to set up the testing software;

FIG. 26 is a flow chart describing the procedure to record the reference activity;

FIG. 27 is a flow chart describing the procedure to perform the neuroactivity testing of the neural cell cultures;

FIG. 28 is a flow chart outlining the basic steps in the control testing procedure;

FIG. 29 is a flow chart describing the procedure to analyze the neuroactivity data;

FIG. 30 is a flow chart describing the procedure to compare the neuroactivity data from the control and metabolite testing procedures to determine if the metabolites have an effect on the neural cells.

### DETAILED DESCRIPTION OF THE INVENTION

While the production and application of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

The present invention takes advantage of mammalian neuronal networks grown on substrate integrated “microelectrode arrays” (MEAs). Primary cultures from dissociated tissue have superior adhesion to the recording substrate, stability during recording, longevity. When primary neuronal cell cultures are grown on MEAs, these devices have a sufficient number of active channels that may be observed on the spike level with good signal-to-noise ratios. These observations include: (1) neuronal networks most likely respond to any substance that has a major effect on central nervous system functions; (2) the sensitivities and efficacies are comparable to those causing responses in vivo; (3) false positives and false negatives are generally minimal and, in many cases, may be predictable; (4) agent response profiles are reproducible and, with changes and/or improvements in data processing, may be used to identify mechanisms and classify an increasing number of substances; and (5) a simple, reliable warning system may be constructed.

“Neuronal Network Biosensors” (NNBS) are living nerve cell networks growing on arrays of substrate integrated miroelectrodes in cell culture. The living nerve cell networks are constantly and spontaneously active and allow long-term (months) monitoring of action potential (AP or “spike”) patterns from as many as 64 channels simultaneously. These living nerve cell networks, as isolated neural tissue, have the advantage of being devoid of the blood-brain barrier and other non-neuronal homeostatic mechanisms that are highly sensitive to their environment and they respond to chemical and physical changes in the life support medium with increases, decreases, or pattern changes in their spike activity. In addition, AP amplitude decreases reflect metabolic changes that lead to a reduction of the membrane potential.

The term “transgene” is used herein to describe genetic material that may be artificially inserted into a mammalian genome, e.g., a mammalian cell of a living animal. The term “transgenic animal is used herein to describe a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal according to methods well known in the art. The term “stem cell” as used herein refers to pluripotent stem cells, e.g., embryonic stem cells, and to such pluripotent cells in the very early stages of embryonic development, including but not limited to cells in the blastocyst stage of development.

A “transgene” is meant to refer to such heterologous nucleic acid, e.g., heterologous nucleic acid in the form of, e.g., an expression construct (e.g., for the production of a “knock-in” transgenic animal) or a heterologous nucleic acid that upon insertion within or adjacent a target gene results in a decrease in target gene expression (e.g., for production of a “knock-out” transgenic animal). A “knock-out” of a gene means an alteration in the sequence of the gene that results in a decrease of function of the target gene, preferably such that target gene expression is undetectable or insignificant.

Transgenic knock-out animals include a heterozygous knock-out of a target gene, or a homozygous knock-out of a target gene. “Knock-outs” as used herein also include, e.g., conditional knock-outs, wherein alteration of the target gene can be activated by exposure of the animal to a substance that promotes target gene alteration, introduction of an enzyme that promotes recombination at the target gene site (e.g., Cre in the Cre-lox system), or other method for directing the target gene alteration.

A “knock-in” of a target gene is used herein to define an alteration in a host cell genome that results in altered expression (e.g., increased or decreased expression) of a target gene, e.g., by introduction of an additional copy of the target gene, or by operatively inserting a regulatory sequence that provides for enhanced expression of an endogenous copy of the target gene. “Knock-in” transgenics include heterozygous knock-in of the target gene or a homozygous knock-in of a target gene and include conditional knock-ins.

Generally, the readout from such systems may be any change from the normal activity that a particular culture has established. Not all networks have identical starting (or native) activity as long as they are spontaneously active. Note that the NNBS does not have to generate exactly the same patterns as the tissue in vivo. It is only necessary to establish a “cultured network correlate response” that can be reliably elicited from networks in response to a certain class of compounds for which the physiological effect is known. For high-throughput application, large numbers of integrated microculture chambers containing a variety of neural and non-neural tissues with a microfluidic system that can mimic normal physiological routing and interactions may be developed, and is expressly part of the invention disclosed herein.

The NNBS is a generic sensor that mimics pharmacologically the nervous system of an animal. For example disinhibitory compounds all enhance bursting and regularization of the burst pattern. Such compounds all cause epilepsy in mammals. Therefore, regularization of burst patterns in cultures and epilepsy may be correlated.

Microelectrode arrays (MEAs) come, e.g., in single and dual network designs. The dual networks provide a control culture that can monitor the life support system or provide a second network. Use of a dual network array allows the growth of “twin networks” that have the same seeding date, seeding pool, and feeding manipulations. Cultures grown on the dual network array grow under the same medium in isolated adhesion areas and are separated into separate medium pools only upon assembly of the chamber. A dual network design may use, e.g., a 5×5 cm plate and edge contact arrangement. Each network may be served by, e.g., 32 microelectrodes.

Burst pattern changes in response to an agent may be recorded as integrated spike data displayed on a chart recorder. The results from different studies may be recorded and cataloged such that the molecular signature of such agent(s) may be used in sampling unknowns. Examples of compounds that may be tested and cataloged include, e.g., mind-altering drugs such as the cannabinoids or even substances that have subtle effects generally detected as tinnitus, hallucinations, vertigo, irritability, loss of concentration, and minor loss of muscle coordination.

Generally, networks with 1,000 to 5,000 neurons growing adhesion areas with 3 to 4 mm diameters may be used. These systems can lose a significant percentage of neurons without showing any deficit in their spontaneous activity or their pharmacological responses.

In operation, neuronal cells over the recording matrix (1 mm² area) and axons from outside the recording matrix supply the spontaneous activity. Despite density fluctuations, a stabilization of neuronal counts past 30 days is obtained. Neuronal losses are approximately 20% in 100 days (6% per month). Neuronal counts include the total number of active signals recorded from the culture. The exclusion of a signal from the count does not signify neuronal cell death, only a loss of activity. NNBS responses are generally histiotypic, that is, the networks act as physiological sensors that can predict the effects of unknown compounds on the nervous system and allow an extrapolation to behavioral deficits.

Furthermore, because the networks express the same receptors and channels found in the parent tissue they have been found to respond very much like the nervous system of an animal would respond. Networks growing in culture on substrate integrated microelectrode arrays serve to link the molecular biochemistry of the network with results from whole animal physiology. The networks of the present invention may be used to provide rapid and accurate information on one or more pharmacological or toxicological changes.

Although a typical network has between 1,000 and 5,000 neurons, the number of inputs in, e.g., a 64-amplifier recording system, limit analysis to 64 sites of the network. Using spike separation, e.g., it is possible to record from more than 100 individual neurons, as many electrodes carry signals from more than one axon. With the present 32 DSPs (digital signal processors), 32 channels may be selected for digitizing. Under optimal separation conditions, a user may record a maximum of 128 active units (4×32). For most sensing uses, however, such a high number of channels is more than sufficient.

Responses to toxicants are usually global, i.e., all channels are affected in a highly similar manner. Such responses can be detected reliably (and be quantified) with data from 10 to 20 channels. Responses to hallucinogens may be more complex by generating unit-specific responses where groups of different neurons respond differently. Therefore, the number of electrodes required to give a statistically sound representation of the network depends on the complexity of the response. Fortunately, in toxicology the end points of many, if not most, responses are relatively simple.

Response Quantification. Response quantification occurs generally in three stages: (1) detection; (2) classification; and (3) identification. Detection will depend on independent multivariate z-scores, i.e., on changes of any activity variable or group of activity variables that exceed 2 or 3 standard deviations of the reference activity. Classification is based on simple, but major physiological responses that will be identified as inhibitory, disinhibitory and excitatory. Whereas inhibition and excitation depend heavily on spike rate, disinhibition (which emerges during generation of epileptiforn activity) requires measurement of pattern regularity. An important distinction between excitation and disinhibition is that both types of responses increase spike production, however, the resulting patterns are radically different. Excitation increases activity without favoring regularity. Disinhibition (substances that silence inhibitory circuits by blocking GABA and or glycine receptors) always generates bursting and high burst pattern regularity.

Identification after classification is a complex task and requires extensive scrutiny of response profiles and application of a variety of methods that have not yet been identified completely. Response profile matching with those generated by known compounds is certainly an essential step. Using the present invention, a number of systems may be tested and quantified for detection, classified and identified. Often, a single unique feature of the profile may identify a compound, e.g., botulinum toxin A. The features of a botulinum Toxin A response includes a long, concentration-independent delay and slow, but irreversible decline of all activity that is highly unique. The delay is caused primarily by receptor dependent internalization of this large protein proenzyme.

Biostatistics. A Plexon MNAP 64 channel workstations using Plexon data acquisition software and the NEX Technologies Neuroexplorer program may be used for data acquisition and analysis. The Plexon system allows action potential (AP or spike) discrimination with 32 digital signal processors that simplify the data before it reaches the host computer. In optimal cases, four different active units could be distinguished per channel resulting in a maximum capacity of 128 logical channels available for analysis.

Normally, the 64 electrode MEA yields an average of 30 channels with good signal-to-noise ratios where at least one or two units can be clearly identified and separated on each channel. The 64 electrode MEA yields an operational maximum of 60 logical channels. Both spike time stamps and waveforms may be collected for analyses of pattern changes and influences on membrane potentials or voltage-gated channel performance that would alter the AP wave shape. Data can be exported to Excel, Kaleidagraph, and Matlab (among many other programs for plotting or further statistical analyses).

The multichannel environment is still somewhat unique in electrophysiology and effective methods for optimal network analyses are evolving. The following basic montage of plots for characterization of the network dynamics may be used: (1) temporal evolution of burst and spike rates in terms of cross channel means and their standard deviations; (2) dose-response curves based on spike production on all channels; (3) temporal evolution of burst variables (a) duration, (b) period, (c) max spike frequencies in bursts; and (d) burst coordination across channels. Because studies can last anywhere from 15 minutes to more than 48 hrs and network responses need to be followed in real-time, it is convenient to form “minute means (MM)” for all burst variables (except rate, which is a scalar) and follow the network responses in terms of one minute steps. These minute means are grouped into “experimental episode means (EEM)” that are then compared to the reference activity mean.

The system is often adjusted for substance-specific effects that can influence the final analysis. Often it is necessary to select a “response stationarity” for best results. For example, synaptic receptor-mediated responses are generally rapid, but often decay as the network adapts or as the substance is enzymatically degraded. Conversely, metabotropic receptor-mediated effects are generally slower in changing network activity, but will reach a maximum effect for a variable period of time. In addition, response times are concentration-dependent. Therefore, in this environment, a fixed-time protocol must be supplemented by selecting periods of network stationarity, where activity establishes a constant pattern.

Therefore “experimental episode means” may be calculated from time periods that are shorter than the episode defined by test substance application to the next medium change.

Networks Statistics. The classical spike train statistics of NEX may be supplemented with more useful network statistics. For example, by using minute means that lead to test episode means, and subsequently cross channel (or network) episode means, and the use of coefficients of variation.

Chip Design. MEAs may be fabricated using, e.g., chromium masks and may be obtained from Photronix, Colorado Springs, Colo. Further customization may be useful for specific applications. MEAs are made often from a rugged glass carrier plate, indium-tin oxide conductors with gold deposits at exposed sites and dimethyl polysiloxane as insulator. MEAs have been found to have a lifetime of several years and are not toxic. MEAs are remarkably rugged, some have been used for 8-10 cycles of use, e.g., 2 months under warm medium for each cycle, followed by autoclaving and flaming to activate the surface before coating with polylysine and laminin, without an appreciable loss of function.

Sample Collection and Preparation. A generic sensor may be designed and used that has the capability to sample water, air (with appropriate concentration and elution steps), and even human serum and urine. The NNBS is combined with a sample and, e.g., a 2× concentrations of supply medium in order to obtain a maximum concentration of a potential toxicant. It may even be feasible to obtain a 25% medium, 75% sample water ratio or even higher concentrations of media depending on the solubility of the basic components of the media and their interaction with the sample.

Flow Rates. Closed chambers often operate at 20 to 40 μl per min. This flow rate is dictated by the small laminar flow chamber design that has only a 300 μm space between the cells and the glass window. Higher rates cause shear stress of cells, channel destabilization and changes in activity. Over long periods of time the shear stress will promote Ca⁺⁺ entry and cell death. As these flow rates are too slow for rapid sample detection, the chambers may be modified to accommodate a flow rate of 1 ml per min. If tubing distances are kept to a minimum (such as 20 cm between sample stores and network and small inner diameter tubing is used (1 mm), then a flow rate of 1 ml/min translates to a sampling time of approximately 38 sec.

In operation, the following conditions may be used in a chamber for use with the present invention, namely:

| | |
|---|---|
| Medium Supply: | 200 ml (2X concentration) |
| Internal Water Supply: | 200 ml |
| Total Medium Supply: | 400 ml |

(A) Flow rate through recording chamber at 20-40 μl/min (2.4 ml/hr)

Total Running Time with medium voided: 181 hrs (7.5 days)

Total Running Time (at 40 μl/min) with medium recirculation at a medium usage (voided) of 10 ml/week: 40 weeks (10 months)

(B) Flow rate of 1 ml/min (in modified chambers)

Total Running Time with medium voided: 400 min

Total Running Time with medium recirculation (10 ml per week used & voided): 40 weeks

The above conditions may or may not take sampling into consideration. For example, samples with potential toxic substances are best avoided prior to sampling. Test samples, however, often need to be circulated for a minimum of about 30-360 min. These parameters may be varied depending on the detection time required for pattern stabilization, classification, and possibly identification.

Constant Bath. It is also possible to perform testing with a constant bath chamber. Medium is placed in the chamber (1 ml or 2 ml, depending on the chamber design). Compound aliquots are added in quantities less than 10 μl, giving whole bath compound concentrations in the pico- to micro- range.

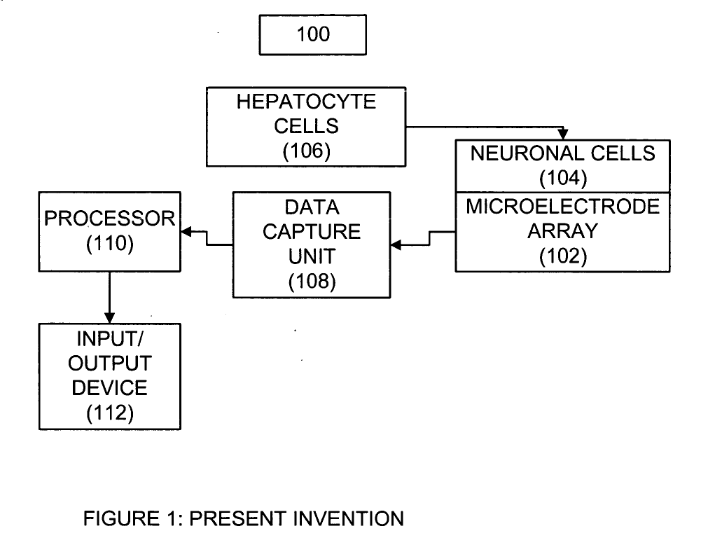

Now referring to FIG. 1, a block diagram of a system 100 in accordance with the present invention is shown. The system 100 for testing the neuronal effects of a compound includes a microelectrode array 102, a data capture unit 108 communicably coupled to the microelectrode array 102, a processor 110 communicably coupled to the data capture unit 108 and one or more input/output devices 112 communicably coupled to the processor 110. The microelectrode array 102, which can be a MEA detector, is capable of supporting wild-type and genetically modified neuronal cells 104 and measuring neuronal activity. The microelectrode array 102 can also be a chamber having a fluid input connected to a perfusion system. The hepatocyte cells 106 are grown in a cell culture flask. The medium from the hepatocyte cells 106 can be extracted and combined, in small amounts, with the medium from the neuronal cells 104. The processor, which can be a computer, compares the neuronal activity of the genetically modified neuronal cells 104 in the presence and absence of the compound.

The system may also include a first and second chamber in fluid communication, wherein the first chamber is separated from the second chamber by a barrier that acts as a blood-brain barrier. The first chamber can be the microelectrode array 102. The neuronal cells can be from a 12-16 day old embryo from a transgenic animal or wild-type animal. The neuronal cells can also be selected from the frontal cortex, the auditory cortex, the visual cortex, the hippocampus or the spinal cord. Furthermore, the neuronal cells may include one or more types of neuronal cells. In addition, the neuronal cells can form a neural tissue. The second chamber can be the hepatocyte cells 106, which can be made from a post-natal animal.

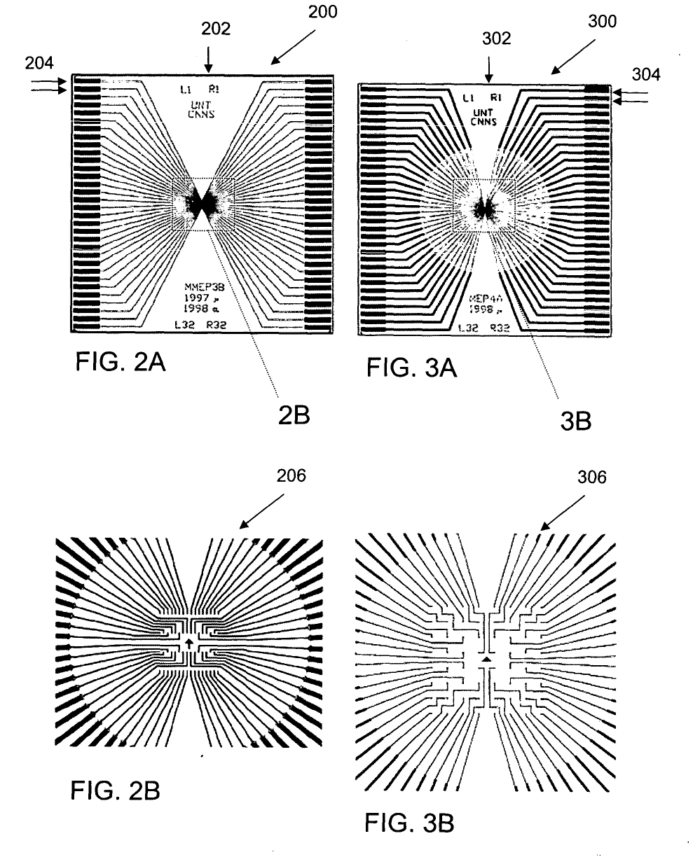

Referring now to FIGS. 2A, 2B, 3A and 3B, typical microelectrode arrays (MEA detectors) 200 and 300 that can be used in connection with the present invention are illustrated. Microelectrode array 200 is a substrate or carrier plate 202 having a number of electrodes within a recording area 206 (FIG. 2B) at the center of the substrate 202. Each electrode is electrically connected to a terminal 204 at the edge of the substrate 202. During use, the terminals are communicably coupled to the data capture unit 106 (FIG. 1). As more clearly shown in FIG. 2B, a 64 conductor MMEP 3B (product of the Center for Network Neuroscience) terminates in a 0.8 mm2 recording area 206 having 4 rows of 16 columns. The electrode spacing is 40 μm between electrodes and 200 μm between rows. The electrode area is roughly 200 μmm2. The carrier plate 202 measures 5×5 cm and is 1.1 mm thick.

Similarly, microelectrode array 300 is a substrate or carrier plate 302 having a number of electrodes within a recording area 306 (FIG. 3B) at the center of the substrate 302. Each electrode is electrically connected to a terminal 304 at the edge of the substrate 302. During use, the terminals are communicably coupled to the data capture unit 106 (FIG. 1). As more clearly shown in FIG. 3B, a 64 conductor MMEP 4A terminates in a 1.2 mm2 recording area 306 having a matrix of 8 rows by 8 columns. Electrode spacing is equidistant at 150 μm. Electrode area is roughly 900 μm2. The carrier plate 302 measures 5×5 cm and is 1.1 mm thick.

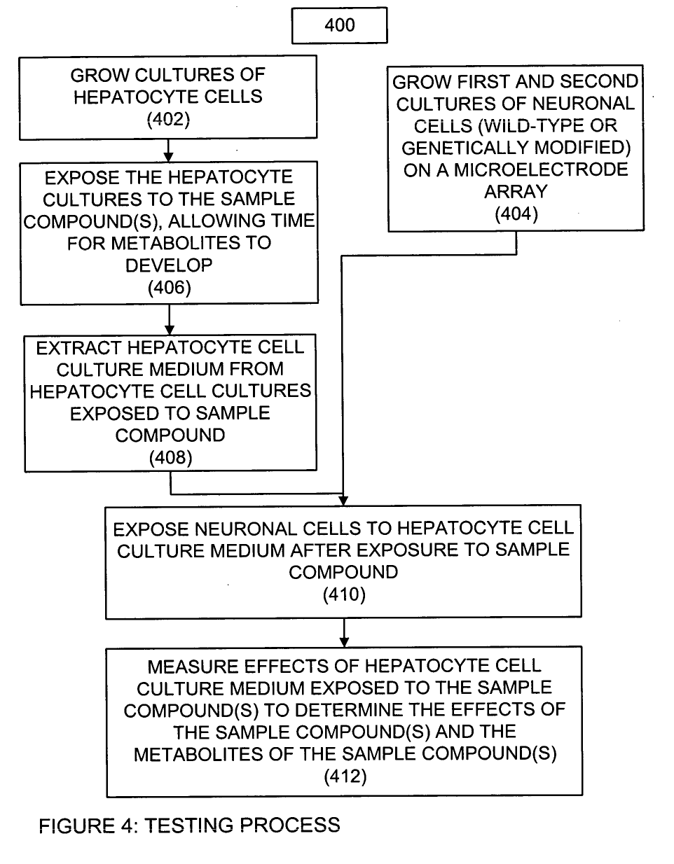

Now referring to FIG. 4, a flow chart illustrating a method 400 of determining the effects of a sample and its metabolites on neuronal cells in accordance with the present invention is shown. A culture of hepatocyte cells is grown in block 402. A portion of the hepatocyte cell cultures are exposed to the sample compound(s) and are given time for the metabolites to develop in block 406. An amount of hepatocyte cell culture medium is extracted from the hepatocyte cultures exposed to the sample compounds(s) in block 408. A first and second cultures of neuronal cells (wild-type or genetically modified) are grown on a MEA in block 404. A portion of the neuronal cell cultures is then exposed to an amount of the hepatocyte cell culture medium that has been exposed to the sample compound(s) in block 410. The effects of the hepatocyte cell culture medium exposed to the sample compound(s) are measured to determine the effects of the sample compound(s) and the metabolites of the sample compounds(s) on neuronal cells in block 412.

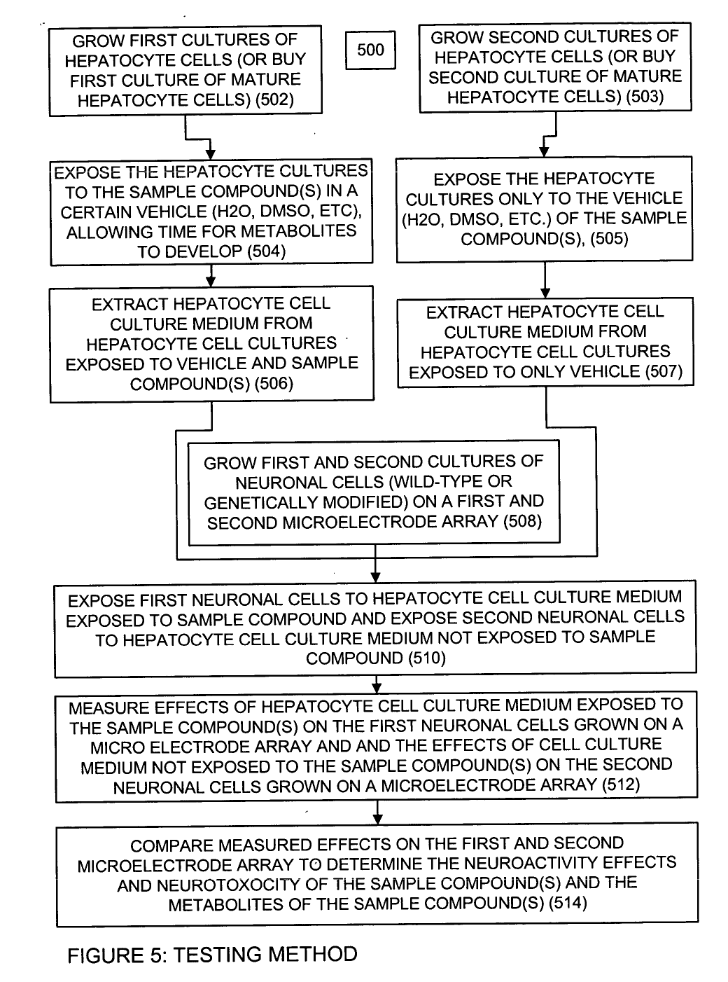

Referring now to FIG. 5, a flow chart illustrating a method 500 of determining the effects of a sample and the metabolites of the sample on a neuronal cell culture in accordance with the present invention is shown. A first hepatocyte cell culture is grown in block 502. The first hepatocyte cell culture is exposed to a sample compound(s) in a delivery vehicle (H₂O, DMSO, etc.) and allowed time for metabolites to develop in block 504. An amount of cell culture medium is extracted from the first hepatocyte culture exposed to the delivery vehicle and sample compound(s) in block 506. A second hepatocyte cell culture is grown in block 503. The second hepatocyte cell culture is exposed to just the delivery vehicle (H₂O, DMSO, etc.) and allowed time for metabolites to develop in block 505. An amount of cell culture medium from the second hepatocyte cell culture exposed to only the delivery vehicle is extracted to be used as a control in block 507. A first and second neuronal cell cultures of neuronal cells (wild-type or genetically modified) is grown on a first and second microelectrode in block 508. The first and second neuronal cells are then exposed to an amount of cell culture medium from a first and second hepatocyte culture, respectively, in block 510. The effects of the amounts of first cell culture medium on the first neuronal cell with the first microelectrode and the amounts of second cell culture medium on the second neuronal cell with the second microelectrode are measured in block 512. The measurements from the first and the second microelectrode are compared to determine the neuroactivity effects and neurotoxicity of the sample and its metabolites on neuronal cell cultures in block 514.

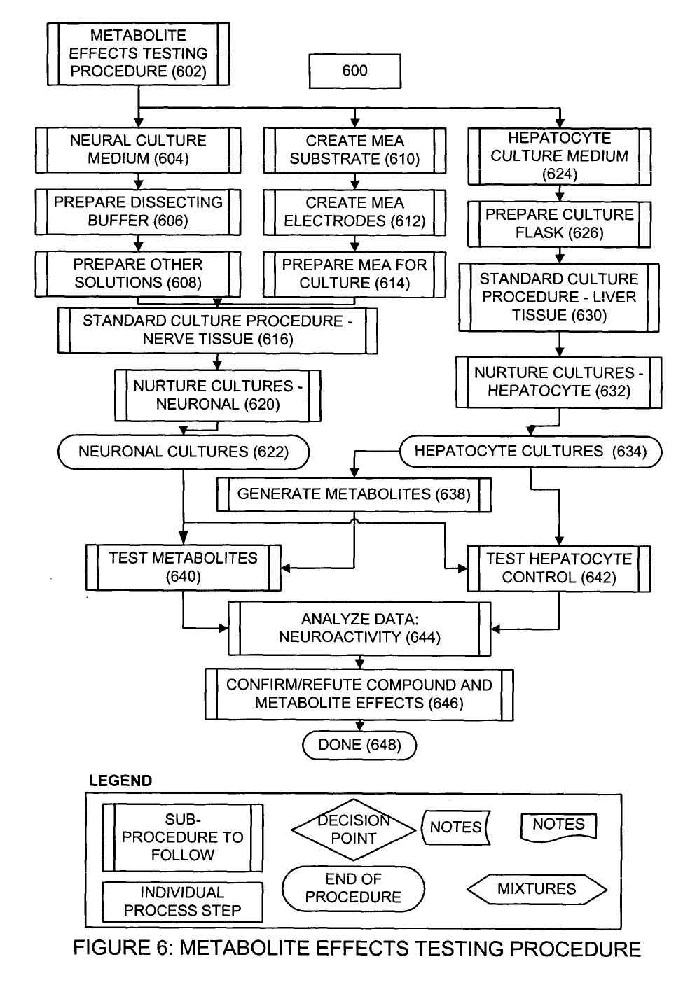

Testing procedures in accordance with various embodiments of the present invention will now be described. Specifically, testing procedures for the metabolite testing procedure (FIG. 6) is described. Hepatocyte cell cultures are grown in cell culture flasks and neuronal cell cultures are grown on microelectrode. Medium from the hepatocyte cell cultures are exposed to a sample compound in its solubility vehicle (experimental) or exposed to only the vehicle (control). The hepatocyte medium is added to the medium of the neuronal cell culture. Neuroactivity data is extracellularly recorded from the neuronal cell cultures. Data from neuronal cell cultures exposed to the experimental and control hepatocyte medium is compared to determine what effects a sample compound and its metabolites have on neuronal cells.

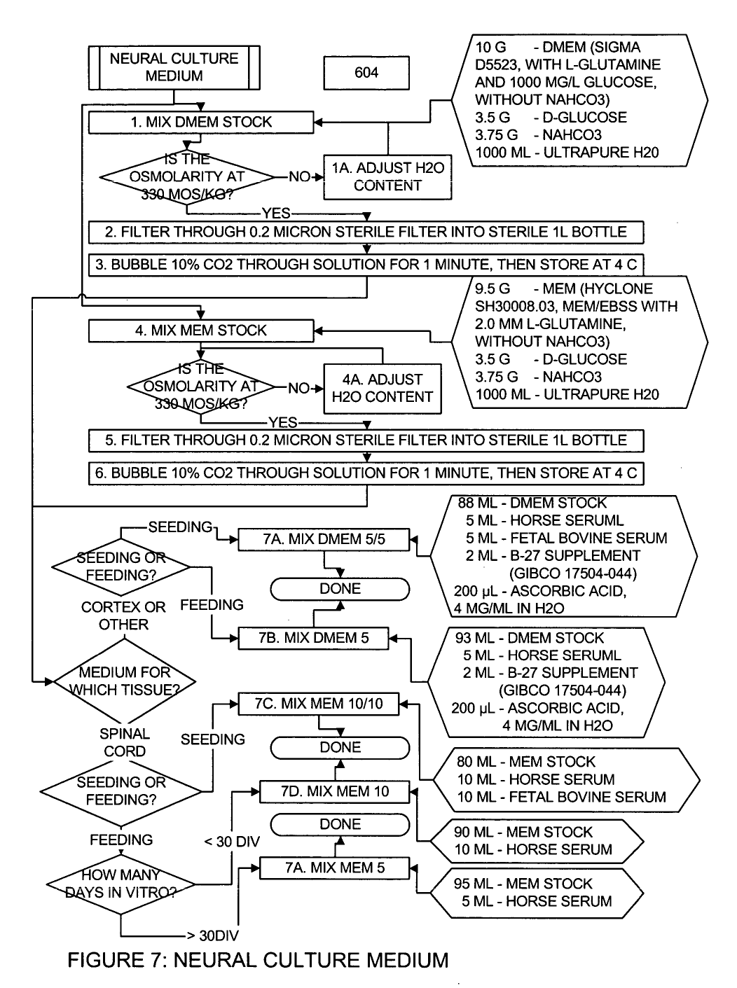

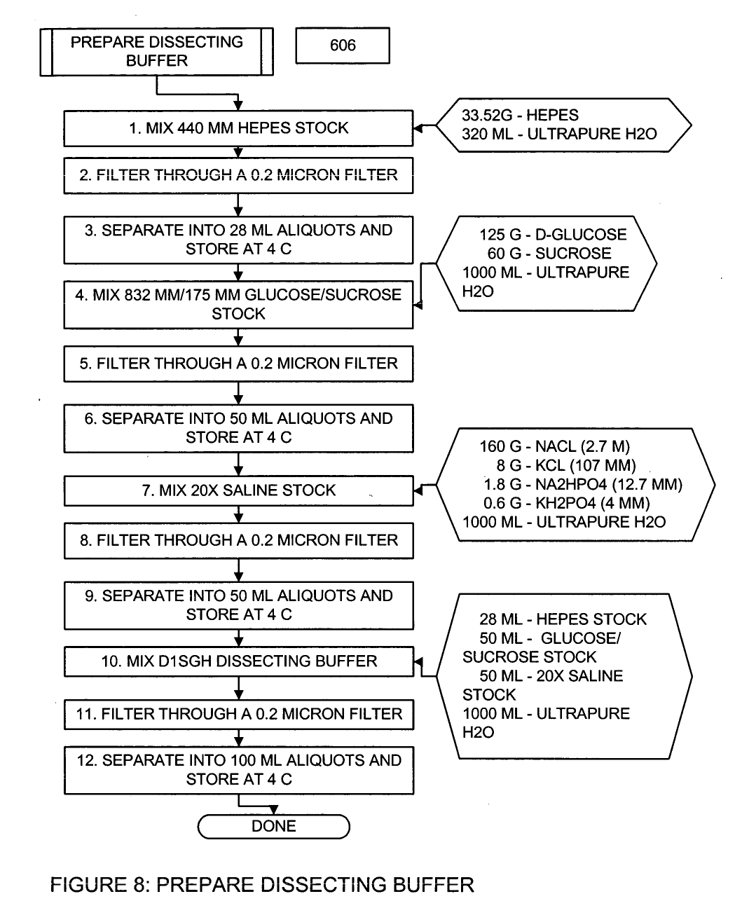

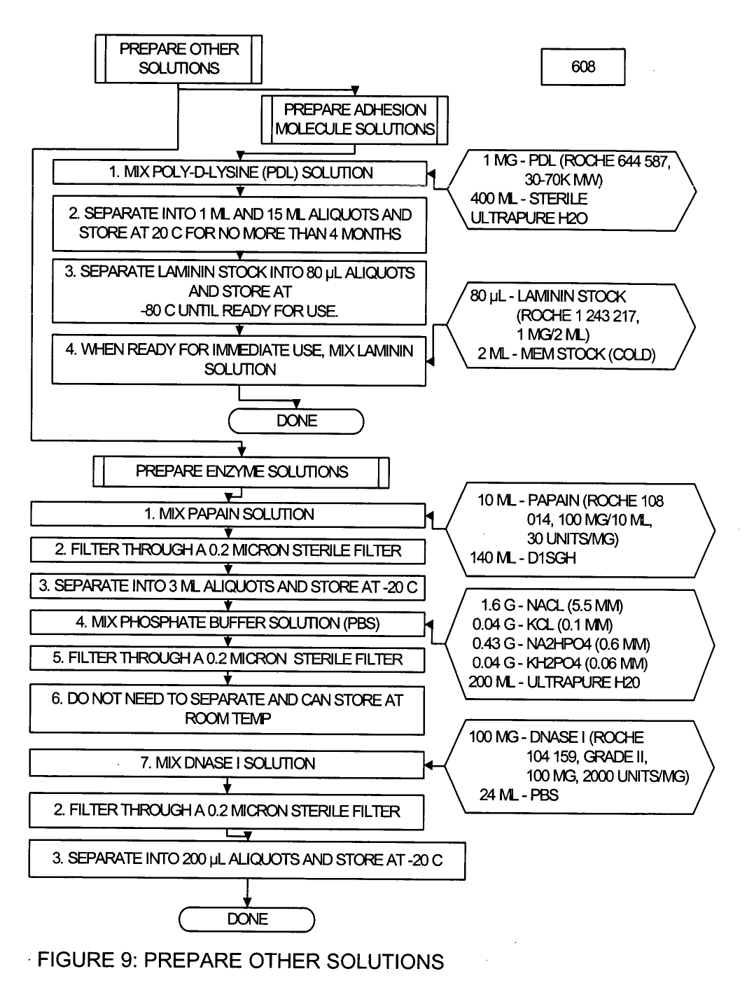

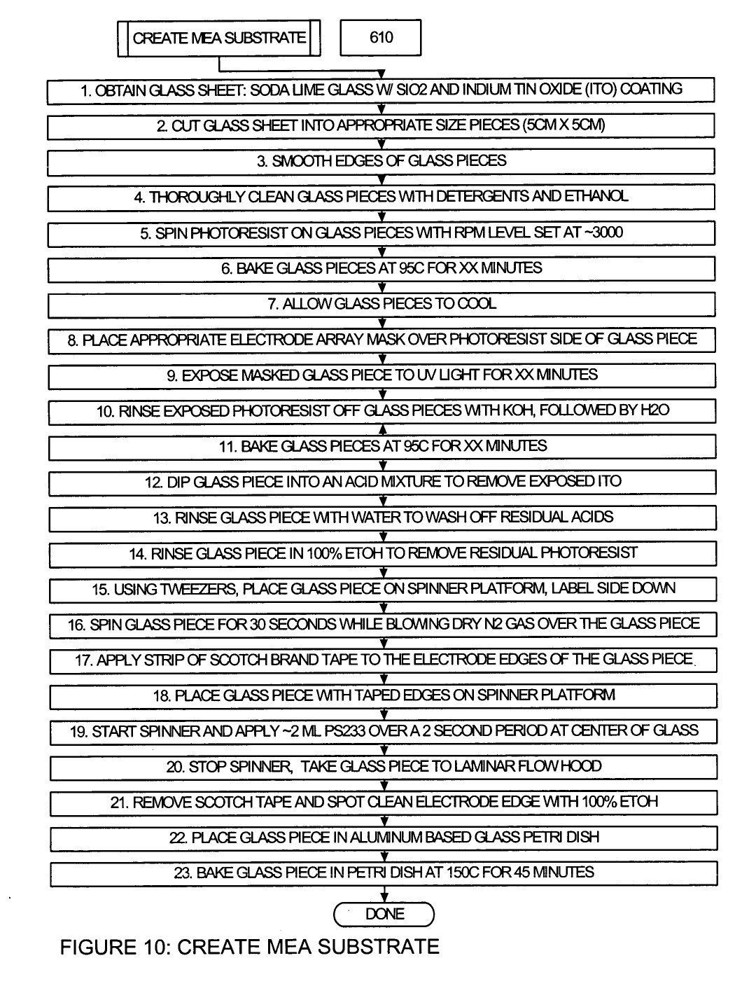

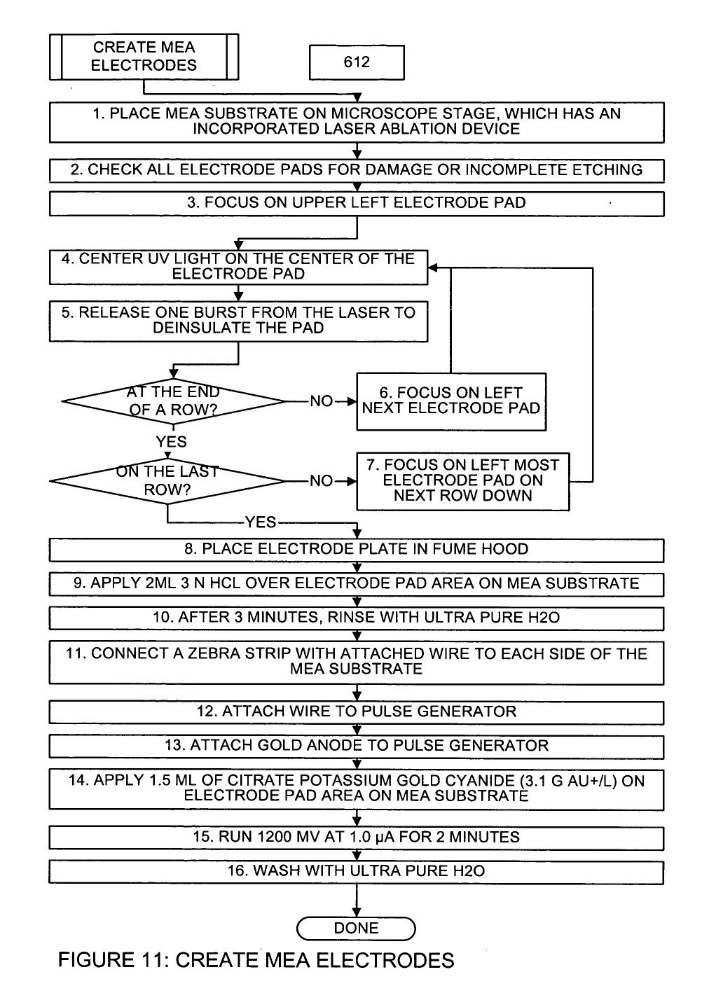

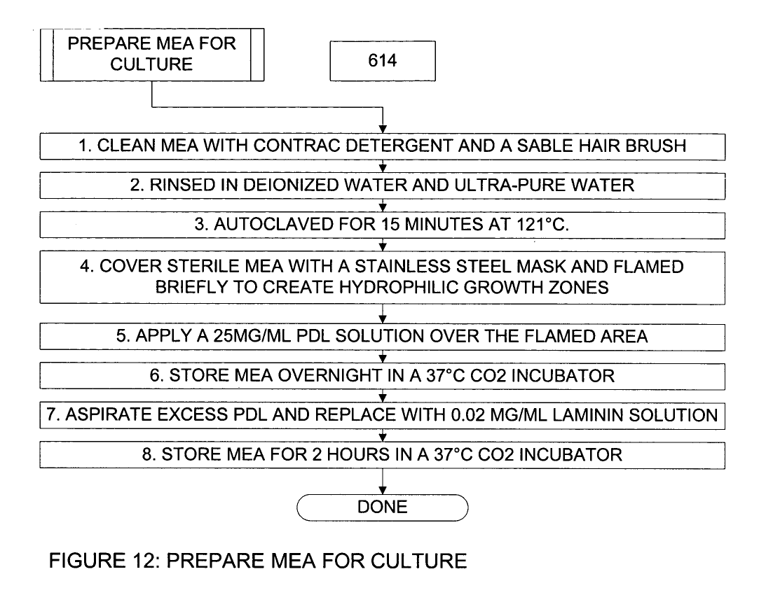

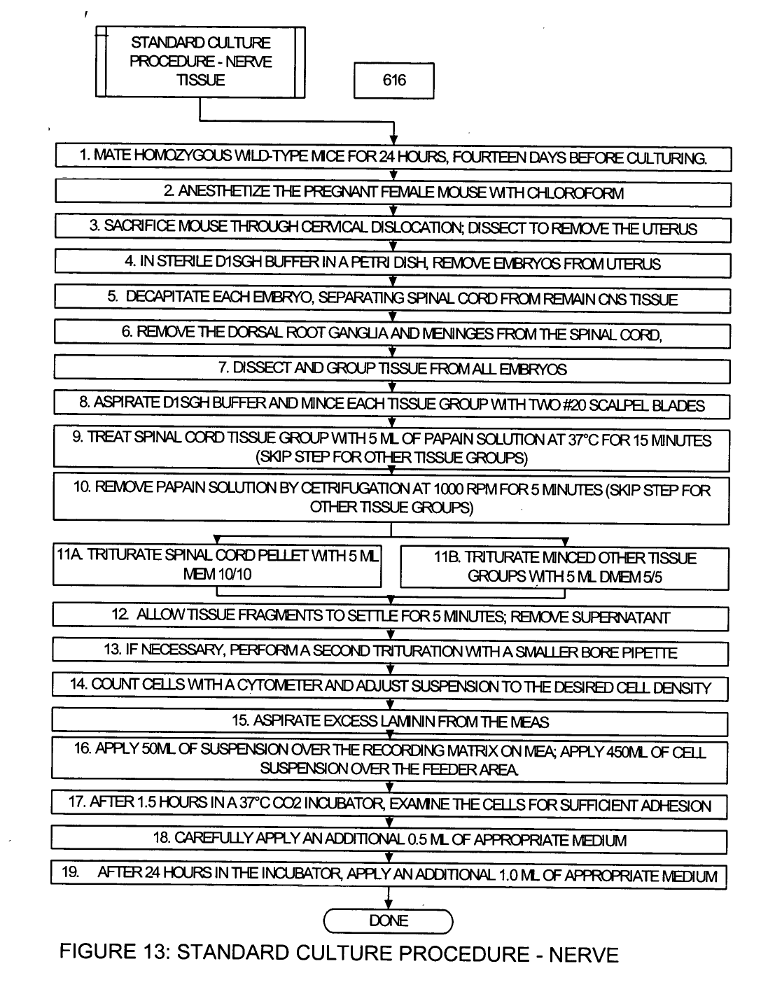

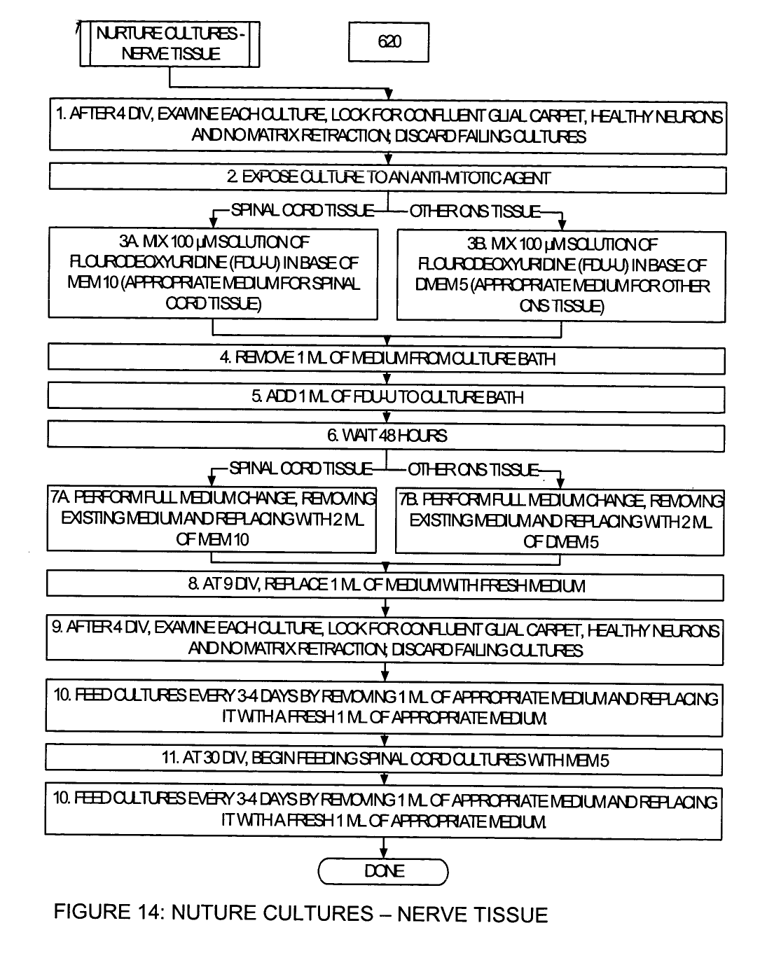

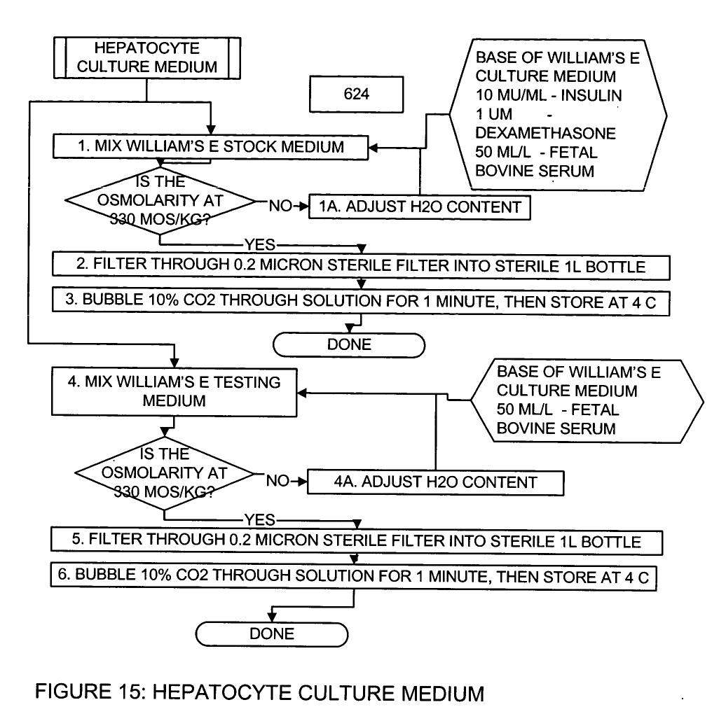

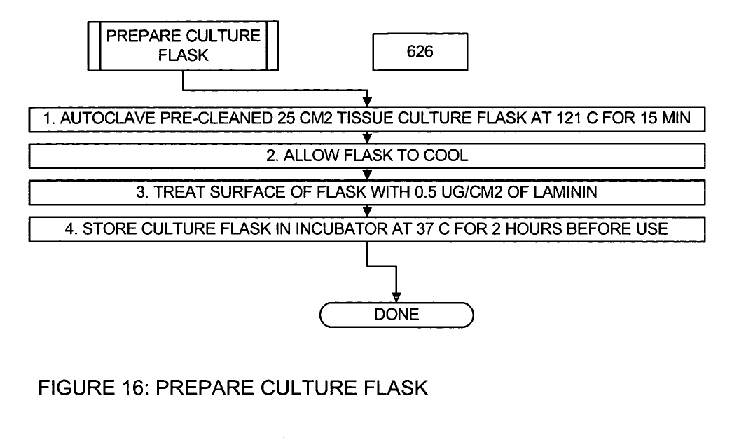

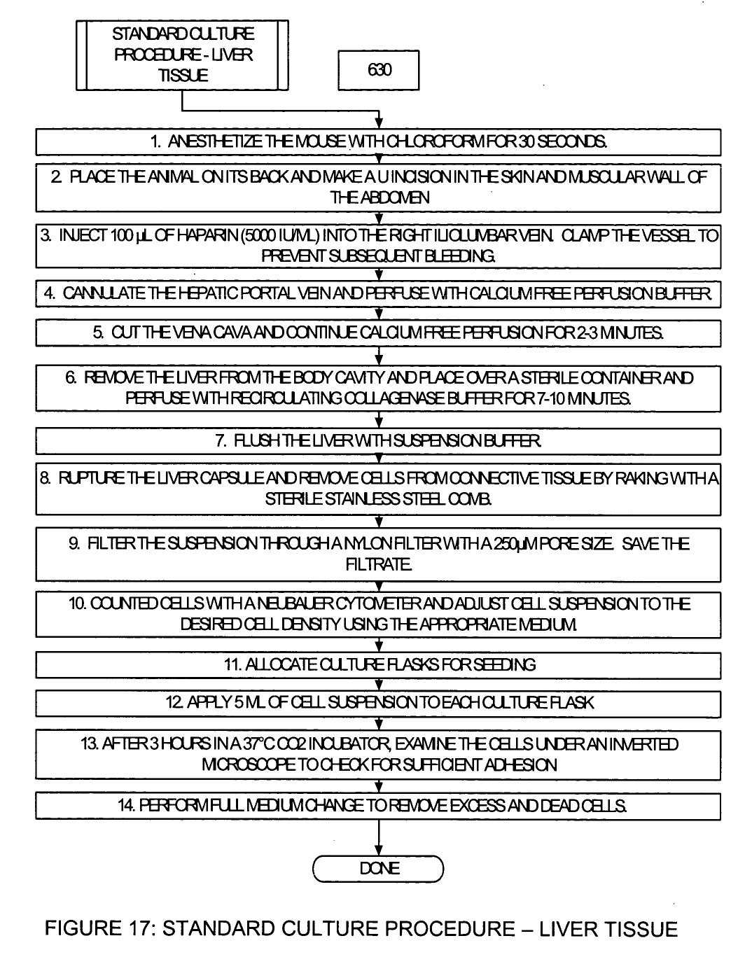

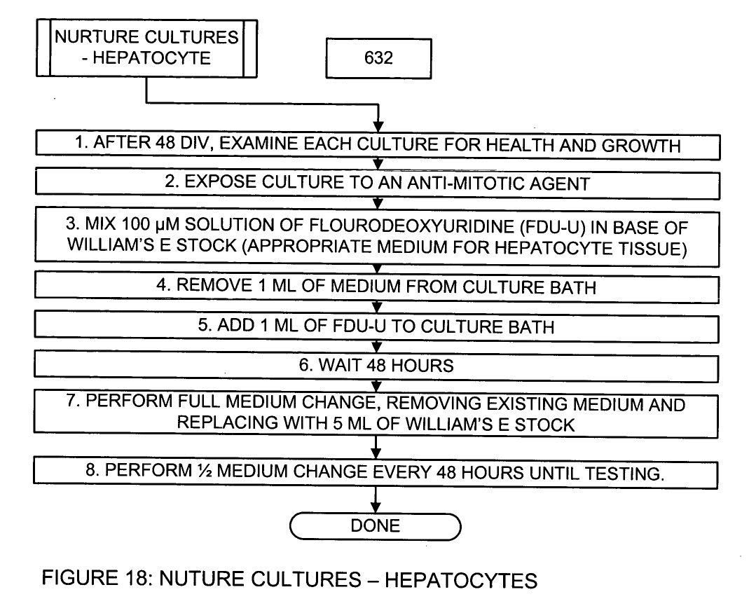

More specifically, the testing procedure for the metabolite testing begins in block 602. Thereafter, a neural cell culture medium is prepared in block 604 (See FIG. 7 and the corresponding description for details), the dissecting buffer is prepared in block 606 (See FIG. 8 and the corresponding description for details) and other solutions are prepared in block 608 (See FIG. 9 and the corresponding description for details). In addition, a MMEP substrate is created in block 610 (See FIG. 10 and the corresponding description for details), the MMEP Electrodes are created in block 612 (See FIG. 11 and the corresponding description for details) and the MMEP is prepared for the culture in block 614 (See FIG. 12 and the corresponding description for details). After the culture medium is prepared in block 604, the dissecting buffer is prepared in block 606, other solutions are prepared in block 608 and the MMEP is prepared for the culture in block 614, the neural cells are cultured in block 616 (See FIG. 13 and the corresponding description for details) and nurtured in block 620 (See FIG. 14 and the corresponding description for details). At the same time, hepatocyte cell culture medium is prepared in block 624 (See FIG. 15 and the corresponding description for details), the hepatocyte cell culture flask is prepared in block 626 (See FIG. 16 and the corresponding description for details), the hepatocyte cells are cultured in block 630 (See FIG. 17 and the corresponding description for details), and the hepatocyte cultures are nurtured in block 632 (See FIG. 18 and the corresponding description for details).

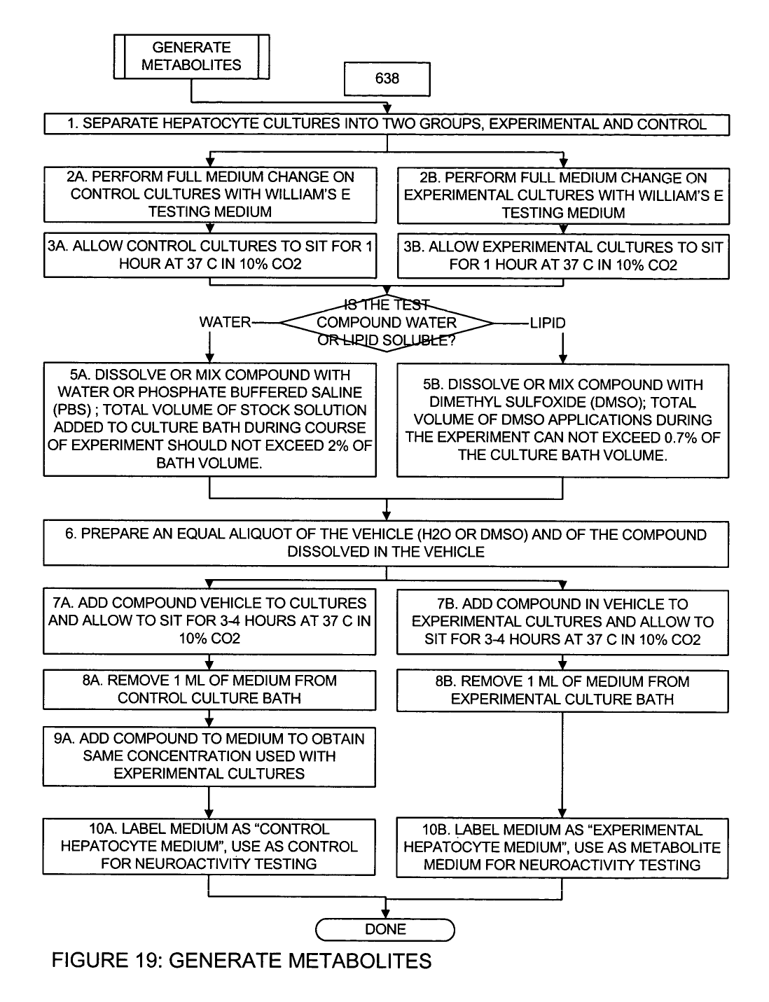

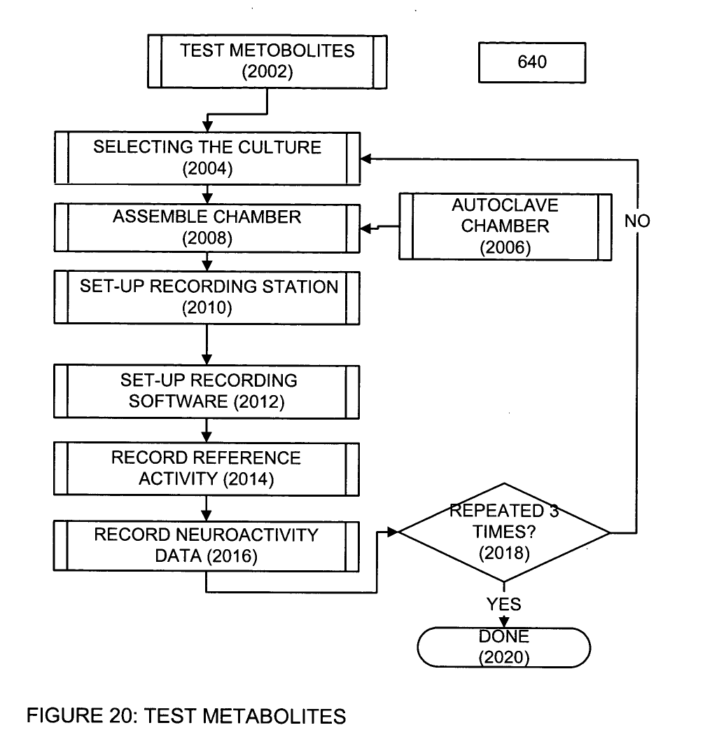

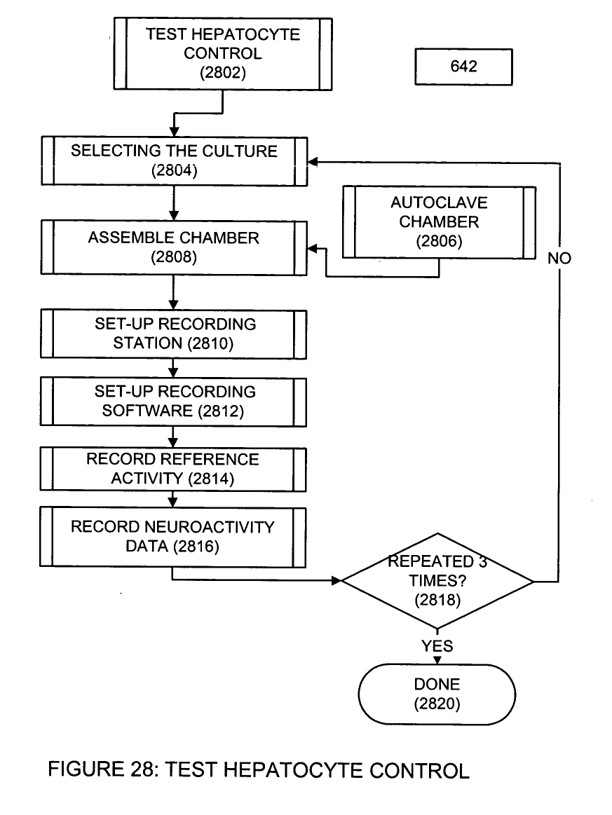

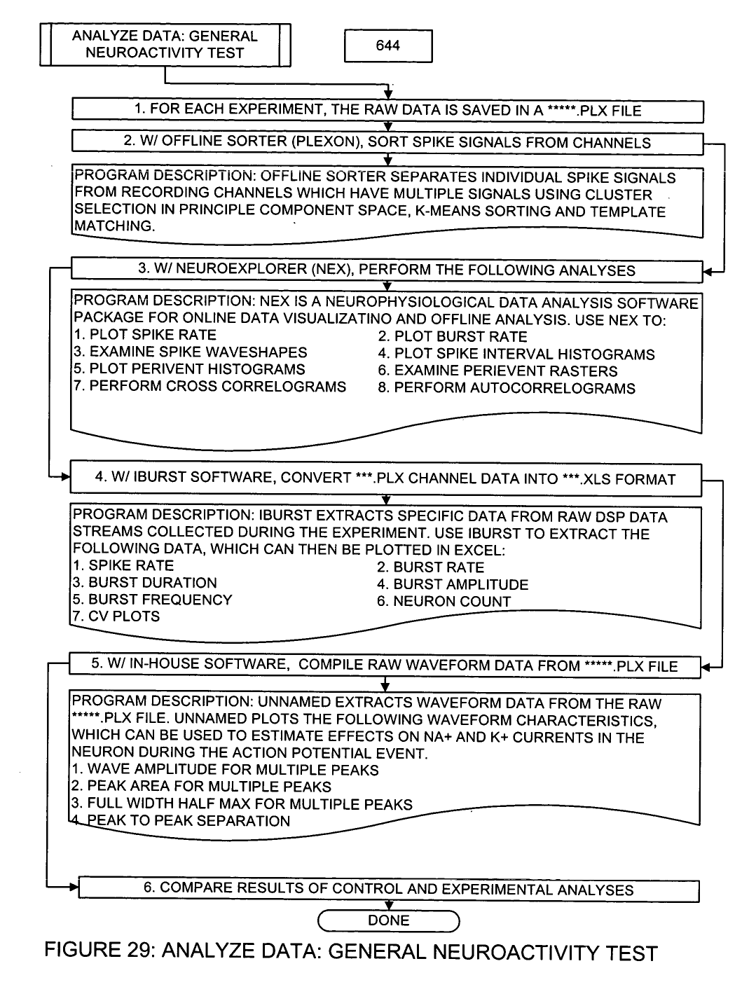

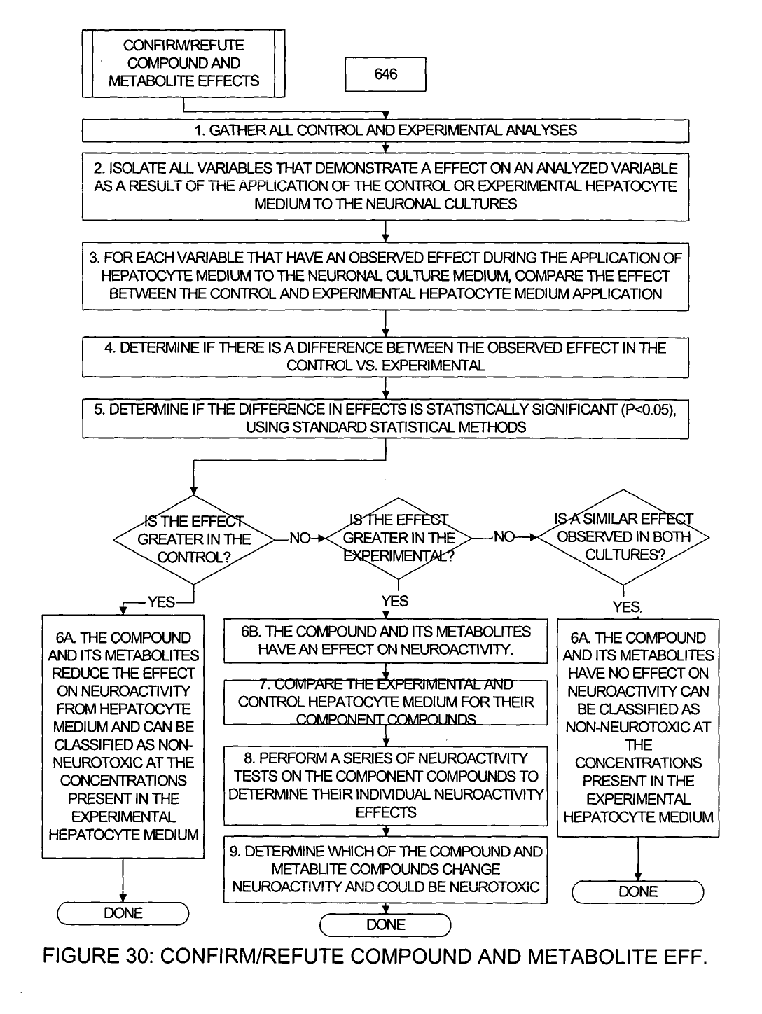

The neuronal cell culture process 604, 606, 608, 610, 612, 614, 616 and 620 produces neuronal cell cultures ready for neuroactivity testing 622. The hepatocyte cell culture process 624, 626, 630, and 632 produces hepatocyte cell cultures ready for testing 634. Hepatocyte cultures 634 are used to generate the metabolites of a sample compound in block 638 (See FIG. 19 and the corresponding description for details). Hepatocyte cell culture medium which includes metabolites from block 638 is tested on neuronal cells 622 in block 640 (See FIG. 20 and the corresponding description for details). Hepatocyte cell culture medium which does not include metabolites is tested on neuronal cells 622 as a control in block 642 (See FIG. 28 and the corresponding description for details). The results from the control 642, and metabolite 640, as tested are analyzed in block 644 (See FIG. 29 and the corresponding description for details). The data from the control 642 and metabolite 640 testing is compared to confirm or refute that a compound and the metabolites of a compound have an effect on neuronal cells in block 646 (See FIG. 30 and the corresponding description for details), thus completing the process in block 648.

Referring now to FIG. 7, the procedure for preparing a nerve cell culture medium 604 (FIG. 6) is shown. Cell culture growth medium is prepared according to the tissue type and stage of maturity of a particular culture. Dulbecco's modified Eagle's medium (DMEM) is prepared for use with frontal cortex and auditory cortex cultures. Cortical cultures are seeded in a mixture of DMEM, 5% horse serum, and 5% fetal bovine serum. After 5 days in vitro (DIV), the fetal bovine serum is removed and the cultures are fed with DMEM and 5% horse serum only. Minimum essential medium (MEM) is prepared for use with spinal cord and hippocampal cultures. These cultures are seeded in a mixture of MEM, 10% horse serum, and 10% fetal bovine serum. After 5 DIV, the fetal bovine serum is removed and the cultures are fed with MEM and 10% horse serum only. After 30 DIV, the horse serum is cut to 5%. Both types of growth medium contain 46 mM sodium bicarbonate as a pH buffer to maintain a pH of 7.4 in equilibrium with an atmosphere containing 10% carbon dioxide.

Now referring to FIG. 8, a flow chart describing the procedure for preparing the dissecting buffer 606 (FIG. 6) is shown. A special buffer solution is prepared to maintain the embryos and tissue during the dissection procedure. The D1SGH dissecting buffer contains HEPES, to maintain a pH of 7.4 in ambient carbon dioxide, glucose to provide metabolic energy to the cells of the embryos once they have been removed from the female, and sucrose and salts to maintain the osmolarity and ionic balance of the cells. This buffer is sterilized and maintained at 4° C.

Referring now to FIG. 9, a flow chart describing the procedure for preparing other solutions (cell adhesion and enzyme solutions) 608 (FIG. 6) is shown. Other solutions are prepared for use in various stages of the procedure. The poly-D-lysine is reconstituted in sterile ultra-pure water and stored at −20° C. and thawed before it is applied to the MMEPs. Laminin is stored at −80° C. in 80 μl aliquots. It is reconstituted in 2 ml cold MEM before it is applied to the MMEPs. The papain solution is a proteolytic enzyme that is reconstituted in D1SGH and stored at −20° C. It is thawed and used in the spinal cord dissociation procedure to facilitate separation of the tissue into single cells. The DNAse solution is reconstituted in physiologic buffered saline and stored at −20° C. It is thawed and used in the dissociation procedure to lyze DNA and histone proteins released from broken cells. These molecules would otherwise cause clumping of the cells and prevent an even monolayer from forming.

Now referring to FIG. 10, a flow chart describing the procedure for creating the microelectrode array (MEA) substrate 610 (FIG. 6) is shown. The microelectrode arrays (MEAs or MMEPs) are created through a standard lithography process. The MMEP is cut from indium tin oxide (ITO) coated soda lime glass. 2 inch by 2 inch pieces of glass are cut and the edges are smoothed. After a thorough cleaning, photo resist is spun on the glass piece and the glass is baked. After cooling, the MMEP mask is placed over the photo resist covered glass and the glass is exposed to UV light. Exposed photo resist is then washed from the glass with KOH and the glass is rinsed with water. The patterned glass is dipped in an acidic solution to remove the exposed ITO. The remaining photoresist is removed with 100% EtOH and the ITO patterned glass is prepared for deposition of the poly-siloxane (PS233) coating by covering the zebra striped edges with tape. PS233 is spun on the patterned glass and baked to harden the PS233 insulation layer.

Referring now to FIG. 11, a flow chart describing the procedure for creating electrodes on the MEA substrate 612 (FIG. 6) is shown. Once the ITO glass is patterned and coated with insulation, it is ready for the electrode process. To create the electrodes, the ITO electrode pads under the insulation layer are exposed with laser ablation. A laser is focused on each electrode pad and fired for a short burst to ablate the insulation layer from the electrode. Once each electrode pad on the MMEP is uncovered, the MMEP is dipped in citrate potassium gold cyanide and the exposed electrode pads are electroplated. A pulse generator is connected to the zebra stripes at the edge of the MMEP to provide the current. Once electroplated, the MMEP is cleaned and is ready for use.

Now referring to FIG. 12, a flow chart describing the procedure for preparing the MEA for cell cultures 614 (FIG. 6) is shown. The MMEP insulation substrate must be prepared to allow the growth of the neuronal network. The surface of the MMEP must be cleaned with a gentle detergent to remove any residue that might inhibit the growth of the cultures, while preserving the integrity of the insulation and maintaining optical clarity. The MMEPs are sterilized by autoclaving and flamed with a butane torch to generate a hydrophilic growth surface. Poly-D-lysine and laminin are applied to promote cell adhesion.

Referring now to FIG. 13, a flow chart describing the procedure for preparing a neuronal cell culture to be used for the neuroacrtivity testing 616 (FIG. 6) is shown. In the standard culture procedure for neuronal cultures, tissue from all embryos is pooled to produce a common cell suspension, which is then seeded on prepared MEAs. Timed pregnant female mice are anesthetized and the embryos are removed. The target tissue is dissected from each embryo and pooled. Spinal cord is treated with a proteolytic enzyme for 15 minutes and then mechanically disrupted into a single cell suspension. Other tissues are mechanically disrupted without enzymatic treatment. The cell suspension is seeded onto the prepared MEAs and allowed to settle for one hour. After one hour, the cultures are filled with 2 ml of medium.

Now referring to FIG. 14, a flow chart describing the procedure for nurturing the neuronal cultures 620 (FIG. 6) is shown. Cultures will be treated to control glial cell growth and maintained for at least one month before experimental use. After 4 days in vitro, cultures are treated with an anti-mitotic agent to prevent the proliferation of glial cells. After 6 DIV, this agent is washed out with a full medium change, and the cultures are fed three times per week subsequently by half medium changes. After one month, the cultures may be used for experiments.

Referring now to FIG. 15, the procedure for preparing hepatocyte cell culture medium 624 (FIG. 6) is shown. William's E stock medium is used for the development phase of the hepatocyte cell cultures. William's E stock includes 10 mU/ml insulin, 1 μM dexamethosone and 5% fetal bovine serum. William's E testing medium does not include insulin and dexamethosone and is used during the metabolite generation process 638 (FIG. 6). William's E Testing medium includes, e.g., 5% fetal bovine serum.

Now referring to FIG. 16, a flow chart describing the procedure to prepare cell culture flask for hepatocyte cultures 626 (FIG. 6) is shown. A flask usually used for cell cultures is cleaned and the desired cell area is covered in laminin.

Referring now to FIG. 17, a flow chart describing the procedure to prepare the hepatocyte cultures for testing 630 (FIG. 6) is shown. An animal is anesthetized and its liver removed. The liver capsule is ruptured and the cells are removed from the connective tissue. The cells are counted in suspension and seeded in a culture flask. After three hours, at 37° C. in a CO₂ incubator, the cells are visually examined for adhesion. A full medium change removes the dead, un-adhered cells.

Now referring to FIG. 18, a flow chart describing the nurturing process for the hepatocyte cultures 632 is shown. A period of time, which could be 48 hours, after seeding, an anti-mitotic agent is added to the cultures. The anti-mitotic agent is removed from the cultures with a full medium change with William's E stock. The cultures are feed with a half medium change every 48-72 hours until testing.

Referring now to FIG. 19, a flow chart outlining the steps required to create the metabolites of a sample compound 638 is shown. The hepatocyte cultures, whether from an animal or an immortalized cell line, are separated into two groups. Both groups receive a full medium change from William's E stock medium to William's E testing medium to remove the insulin and dexamethasone, which are toxic to neuronal cells. After a period, about 1 hour, the sample compound is prepared in a vehicle, which could be H₂O or DMSO, and added to the first group of hepatocyte cultures. An equal amount of vehicle is added to the second group of hepatocyte cultures. After a period of time, which could be 3 hours, an amount of medium is removed from each culture. Medium extracted from the first group is medium containing metabolites and medium extracted from the second group is the control medium.

FIG. 20 describes the procedure to test the metabolite medium on the neuronal network for changes in neuroactivity induced by exposure to the sample compound and its metabolites. The testing process starts with the selection of a neuronal culture and the preparation of the testing chamber. Once the testing chamber is installed into the test station, reference activity is recorded to establish the baseline neuroactivity of the culture. Every culture forms slightly different network connections and therefore has different levels of spontaneous activity. However, each network is capable of responding to a pharmacological agent in a representative manner. Once a base line is recorded, the test compound, which could be the metabolite or control medium, is applied to a neuronal culture, and changes in the cultures neuroactivity are recorded. If the metabolite and control medium elicit a different response from the neuronal cultures, then it signifies that the sample compound and its metabolites have different effects on the cultures than does the sample compound alone. These different effects can be attributed to the metabolites of the sample compound or the hepatocytes ability to process the compound. Changes in each culture's individual base line are compared, and analyzed with in-house and commercially available software and tested for statistical significance (using the standard t-test or other appropriate statistics).

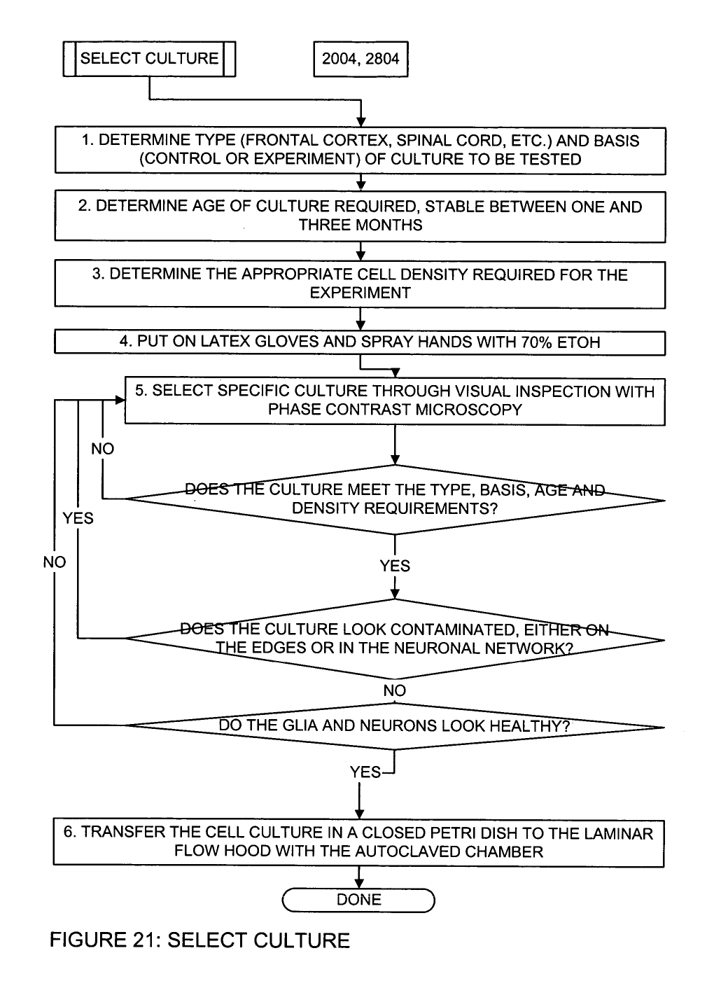

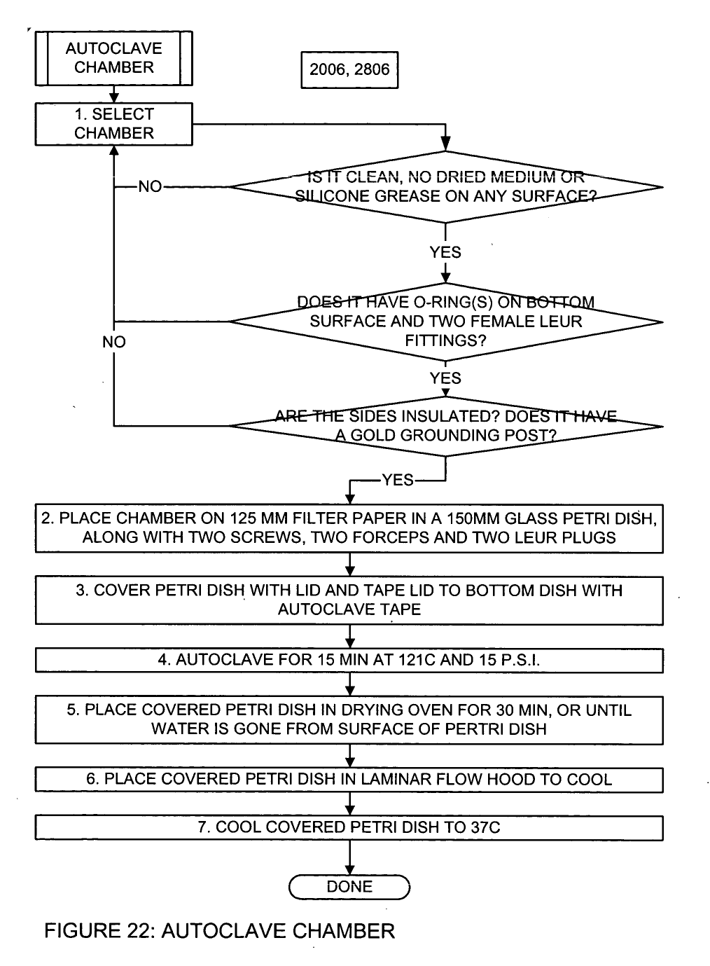

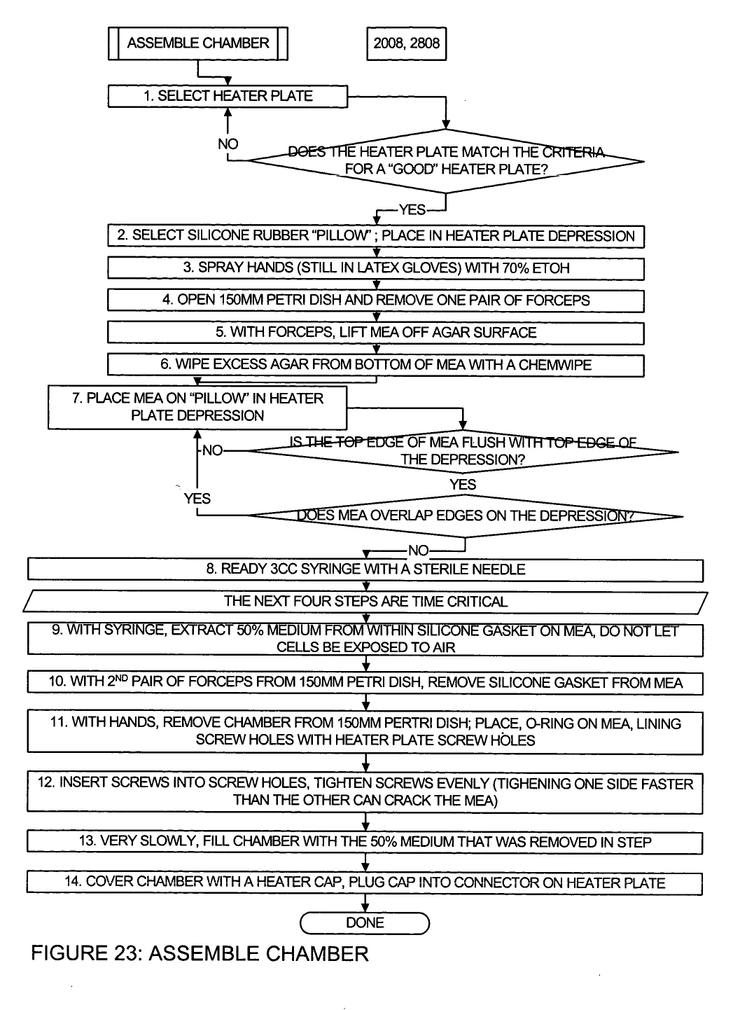

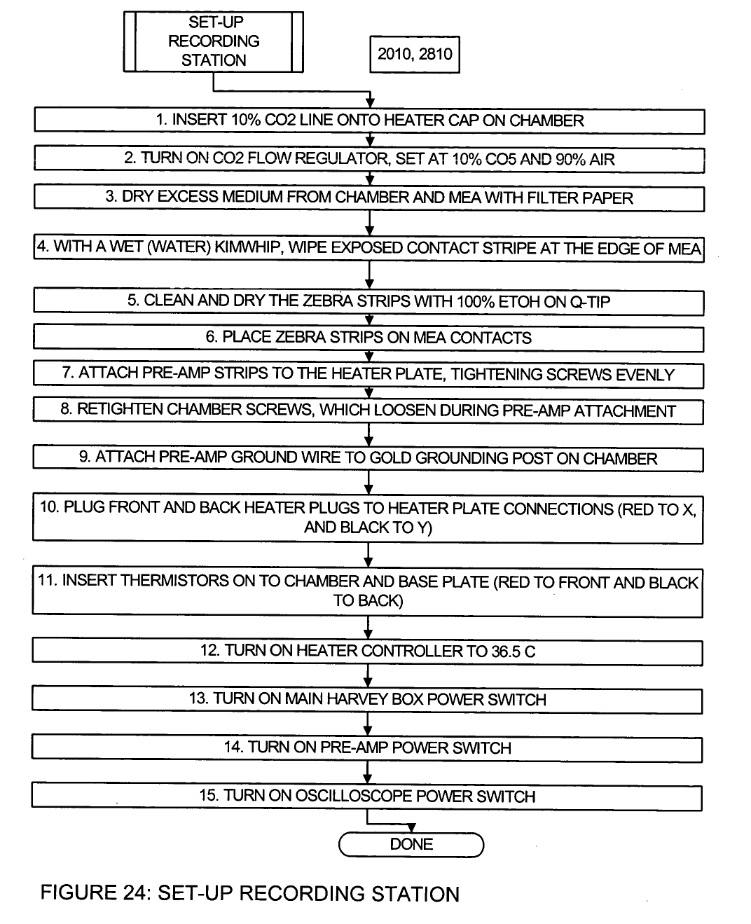

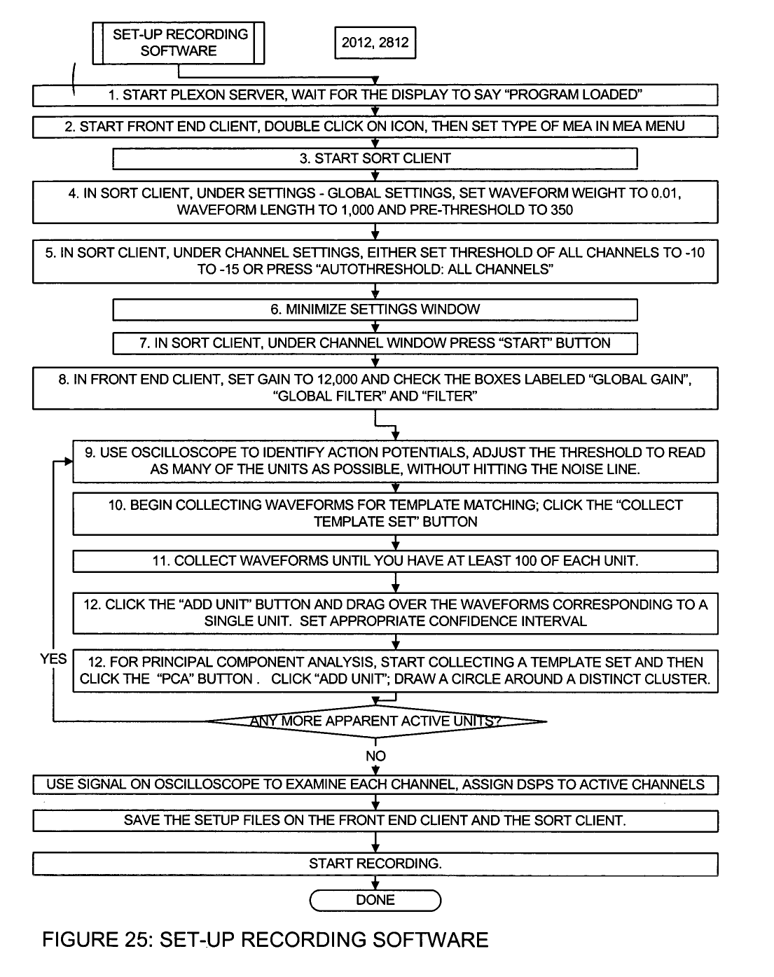

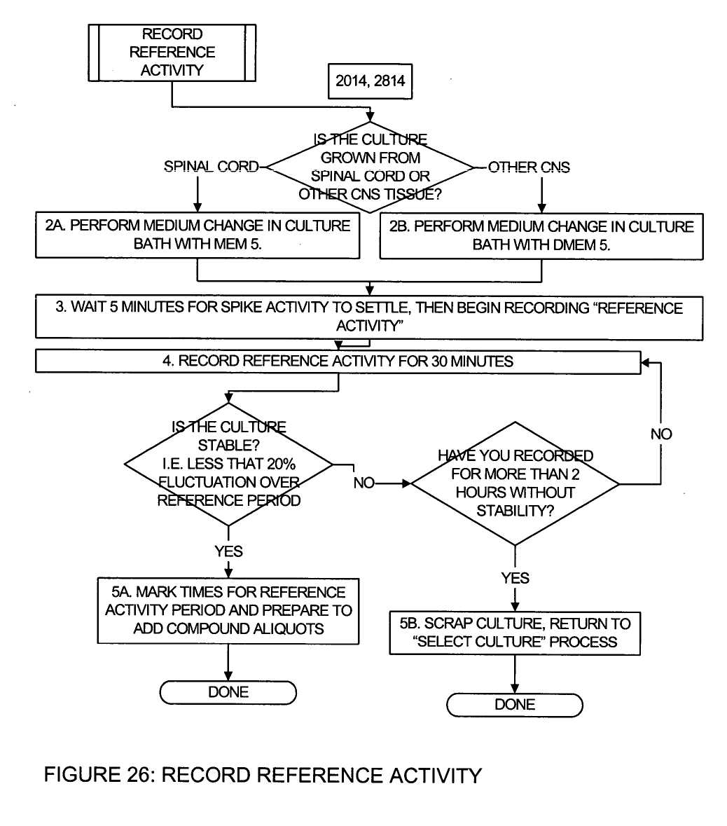

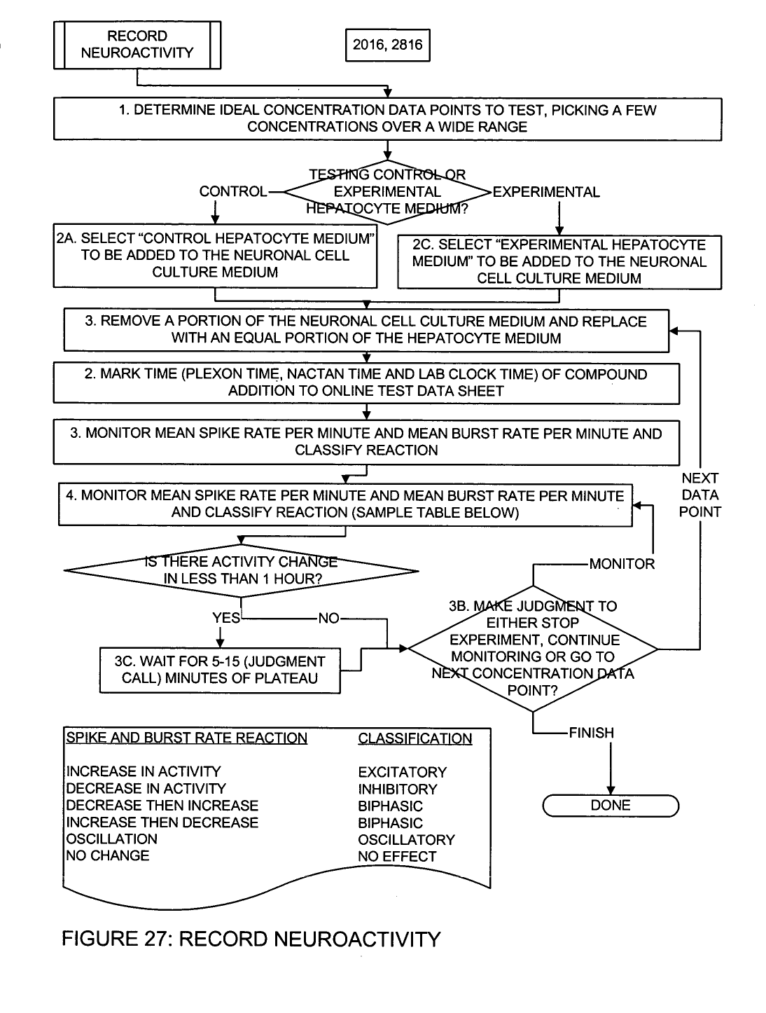

More specifically, the metabolite medium neuroactivity testing 640 (FIG. 6) begins in block 2002 in FIG. 20. The procedure begins with selecting the culture 2004 (See FIG. 21 and the corresponding description for details) and autoclaving the testing chamber 2006 (See FIG. 22 and the corresponding description for details). Once those steps are complete, the testing chamber is assembled in block 2008 (See FIG. 23 and the corresponding description for details). The recording station is setup in block 2010 (See FIG. 24 and the corresponding description for details), the recording software is setup in block 2012 (See FIG. 25 and the corresponding description for details) and the reference activity is recorded in block 2014 (See FIG. 26 and the corresponding description for details). Neuroactivity data is recorded from the neuronal cultures exposed to the metabolite medium in block 2016 (See FIG. 27 and the corresponding description for details). The process beginning at block 2004 is repeated until three data points are obtained for each test, as determined in decision block 2018. The process ends in block 2020

Referring now to FIG. 21, a flow chart describing the procedure to select the cell culture to be used for testing 2004 and 2804 (FIGS. 20 and 28) is shown. An appropriate culture must be selected for the experiment. A culture must meet certain criteria before it may be selected for use in a study. After the appropriate tissue type is selected, a culture that is between one and three months old is chosen. These cultures are visually inspected under a phase contrast microscope to determine if the density of cells is adequate and that the cells are healthy.

Now referring to FIG. 22, a flow chart describing the procedure to autoclave the testing chamber 2006 and 2806 (FIGS. 20 and 28) is shown. Recording chambers must be selected and sterilized prior to use. An appropriate recording chamber must be selected and determined to be clean and in proper working order before it is used. The selected chamber must be sterilized by autoclaving at 121° C. for 15 minutes at 15 p.s.i. The chamber must then be dried in a 70° C. oven and allowed to cool to no more than 37° C.

Now referring to FIG. 23, a flow chart describing the procedure to assemble the testing chamber 2008 and 2808 (FIGS. 20 and 28) is shown. Once the recording chamber has been sterilized and the culture has been selected, the recording chamber, MMEP with cell culture and base plate are assembled into one unit. After an appropriate base plate and MMEP silicone rubber pillow is selected, all pieces are placed in a laminar flow hood. Lift MMEP from petri dish and place on pillow, which is one the base plate. Very quickly, remove half of the medium from the silicone gasket that is over the cells, remove the silicone gasket, put the chamber over the gasket's previous location and add the removed medium to the opening in the chamber over the cells. Cover the open chamber with a heater cap and move the base plate and chamber to the testing station microscope stage.

Referring now to FIG. 24, a flow chart describing the procedure to set up the testing station 2010 and 2810 (FIGS. 20 and 28) is shown. Once on the testing station microscope stage, the base plate and chamber are connected to the system and prepared for testing. A 10% CO₂ line is plugged into the heater cap. Excess medium is dried from the base plate with filter paper. The zebra stripes are wiped clean with EtOH before the pre-amplifiers are attached and clamped down. Grounding wires, heating wires and thermistors are plugged into the base plate and pre-amplifier. The heater controller is set to 36.5° C. and the electronic components are turned on, including the Plexon system and the oscilloscope.

Now referring to FIG. 25, a flow chart describing the procedure to set up the testing software 2012 and 2812 (FIGS. 20 and 28) is shown. Once the biological components are connected to the data acquisition electronics, the software can be set up and the active channels can be identified. The Plexon data acquisition software is loaded, as well as other monitoring and analysis programs. Using the standard Plexon procedures, active electrodes are identified and DSP's are assigned to individual waveform patterns. Each electrode could have as many as four individual waveform patterns, representing different nerve cell signals. Once all of the active units are identified and the DSP's assigned, the data recording starts.

Referring now to FIG. 26, a flow chart describing the procedure to record the reference activity 2014 and 2814 (FIGS. 20 and 28) is shown. The beginning of every testing includes a recording at least 30 minutes of reference activity after a medium change. The medium, the type will depend upon the type of tissue used to create the cell culture, should be replaced in small increments to minimize any turbulence effects from the liquid movement in the chamber. After a full replacement of medium, reference activity recording begins, and ends after 30 minutes of statistically stable activity. If statistically stable activity can not be obtained within two hours, the culture is generally scrapped and a new culture prepared for testing.

Now referring to FIG. 27, a flow chart describing the procedure to perform the testing needed to record the neuroactivity data 2016 and 2816 (FIGS. 20 and 28) is shown. Medium or supernatant, whether control, which includes medium extracted from a hepatocyte culture and combined with an amount of sample compound; or test, which includes medium extracted from hepatocyte cultures exposed to a sample compound, is added to the neural cultures in the test station. A series of concentrations are selected over a wide range. Reactions may take up to 2 to 3 hours to occur. Neuroactivity changes from reference are recorded and defined as either excitatory, inhibitory, biphasic, oscillatory or no effect.

Referring now to FIG. 28, a flow chart outlining the basic steps in the control testing procedure is shown. Control medium testing 642 (FIG. 6) is performed either concurrently or superceding the metabolite medium testing. Control medium neuroactivity testing 642 (FIG. 6) begins in block 2802. The procedure begins with selecting the culture 2804 (See FIG. 21 and the corresponding description for details) and autoclaving the testing chamber 2806 (See FIG. 22 and the corresponding description for details). Once those steps are complete, the testing chamber is assembled in block 2808 (See FIG. 23 and the corresponding description for details). The recording station is setup in block 2810 (See FIG. 24 and the corresponding description for details), the recording software is setup in block 2812 (See FIG. 25 and the corresponding description for details) and the reference activity is recorded in block 2814 (See FIG. 26 and the corresponding description for details). Neuroactivity data is recorded from the neuronal cultures exposed to the metabolite medium in block 2816 (See FIG. 27 and the corresponding description for details). The process beginning at block 2804 is repeated until three data points are obtained for each test, as determined in decision block 2818. The process is completed in block 2820.

Now referring to FIG. 29, a flow chart describing the procedure to analyze the data 644 (FIG. 6) from both the metabolite and control medium neuroactivity testing. The extracellular recording data is stored in a *.plx file, from Plexon, Dallas, Tex. The data consists of a series of time stamps and corresponding volt measurements for each recorded channel, as consistent with this type of technology. The data is processes by in-house and publicly available software to extract information on the spike rate, burst rate, number of bursting neurons, wave form, burst amplitude, and other variables versus time.

The final step in the process is to compare the data to confirm or refute a neuroactivity effect 646 (FIG. 6) from the metabolites of a sample compound. FIG. 30 outlines this step. For each experiment, the data from a representative time segment from the reference activity period (after full medium change), or native activity period (no medium change before testing) depending on the protocol, is compared to the data from a representative segment from the medium application period. This difference defines the effect for each type of medium, experimental and control. If there is a statistically significant difference between the effect induced by the experimental medium and the control medium, then the following may be true. If the effect is greater in the control, then the compound has a greater effect on neuroactivity than its metabolites. If the effect is greater in the experimental, the metabolites of a sample compound have a greater effect than the compound alone. If there is no difference, then the sample compound and its metabolites, and the sample compound alone have a same effect on neuroactivity.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A method for determining the effects of a compound and on a neuronal cell comprising the steps of:
obtaining a first and a second hepatocyte supernatant, wherein the first hepatocyte supernatant comprises a supernatant from a hepatocyte exposed to a compound;
exposing a first and second neuronal cell on a first and a second microelectrode, respectively to the first and second hepatocyte supernatants, respectively; and
detecting the effects of the first and second hepatocyte supernatants on the first and second neuronal cells with the microelectrodes, wherein a comparison of the measurements from the first and the second microelectrodes are used to determine the effects of the hepatocyte supernatants on neuronal cells.

2. The method of claim 1, wherein the neuronal cell comprises an embryonic stem cell from a knock-out, knock-in, over-expressing transgenic, under-expressing-transgenic, a conditional knockout, a mutant and the like.

3. The method of claim 1, wherein the neuronal cell is from an animal knock-out, knock-in, over-expressing transgenic, under-expressing-transgenic, a conditional knockout, a mutant and the like.

4. The method of claim 1, wherein the neuronal cells are selected from the frontal cortex, the auditory cortex, the visual cortex, the hippocampus or the spinal cord.

5. The method of claim 1, wherein the heptatocyte cells are selected from an wild-type animal, a genetically modified animal or an immortalized cell line.

6. The method of claim 1, wherein the hepatocyte cell is from an animal knock-out, knock-in, over-expressing transgenic, under-expressing-transgenic, a conditional knockout, a mutant and the like.

7. The method of claim 1, wherein the neuronal cells or the hepatocyte cells include one or more types of neuronal or hepatic cells, respectively.

8. The method of claim 1, wherein the neuronal cells or hepatocyte cells form a portion of a neural tissue or hepatic tissue, respectively.

9. The method of claim 1, wherein the hepatocyte supernatant comprises both the compound and hepatic metabolites of the compound.

10. The method of claim 1, wherein the hepatocyte supernatant comprises hepatic metabolites of the compound.

11. A method for determining the effects of a compound and the metabolites of the compound on a neuronal cell comprising the steps of:
growing a first and second hepatocyte cell culture a compound, wherein the first hepatocyte cell culture is exposed to a compound;
obtaining the medium from the first and second hepatocyte cell cultures;
applying the medium from the first and second hepatocyte cell cultures, respectively, to a first and a second neuronal cell grown on first and second microelectrodes;
measuring the activity of the first neuronal cell with the first microelectrode and the second neuronal cell with the second microelectrode; and
comparing the measurements from the first and the second microelectrodes to determine the effects of the medium on the neuronal cells.

12. The method of claim 11, wherein the medium comprises the compound and the compound's metabolites.

13. The method of claim 11, wherein the medium comprises the compound's metabolites.

14. The method of claim 11, further comprising the step of extracting a supernatant from the medium.

15. The method of claim 11, wherein the medium is cell-free.

16. The method of claim 11, wherein the neuronal cell comprises an embryonic stem cell from a knock-out, knock-in, over-expressing transgenic, under-expressing-transgenic, a conditional knockout, a mutant and the like.

17. The method of claim 11, wherein the neuronal cell is from an animal knock-out, knock-in, over-expressing transgenic, under-expressing-transgenic, a conditional knockout, a mutant and the like.

18. The method of claim 11, wherein the neuronal cells are selected from the frontal cortex, the auditory cortex, the visual cortex, the hippocampus or the spinal cord.

19. The method of claim 11, wherein the heptatocyte cells are selected from from an wild-type animal, a genetically modified animal or an immortalized cell line.

20. The method of claim 11, wherein the hepatocyte cell is from an animal knock-out, knock-in, over-expressing transgenic, under-expressing-transgenic, a conditional knockout, a mutant and the like.

21. The method of claim 11, wherein the neuronal cells or the hepatocyte cells include one or more types of neuronal or hepatic cells, respectively.

22. The method of claim 11, wherein the neuronal cells or hepatocyte cells form a portion of a neural tissue or hepatic tissue, respectively.
